SEQUENCE LISTING (2 sequences)

SEQ ID NO: 1 - DNA, 20 bp, synthetic construct
  source 1..20: mol_type = other DNA
  variation 11: note = n is C or T
  variation 12: note = n is A or C
agagtttgatnntggctcag

SEQ ID NO: 2 - DNA, 21 bp, synthetic construct
  source 1..21: mol_type = other DNA
  variation 5: note = n is C or T
acggntaccttgttacgactt

(19) United States
(12) Patent Application Publication
(10) Pub. No.: US 2026/0232786 A1
(43) Pub. Date: Aug. 13, 2026

(54) ANTIMICROBIAL ACTIVITY OF GUT BACTERIA IN EARLY LIFE

(71) Applicant: Washington University, St. Louis, MO (US)

(72) Inventors: Rodney D. Newberry, St. Louis, MO (US); Sreeram Udayan, St. Louis, MO (US)

(21) Appl. No.: 19/540,271

(22) Filed: Feb. 13, 2026

Related U.S. Application Data
(60) Provisional application No. 63/758,329, filed on Feb. 13, 2025.

(51) Int. Cl. A61K 39/09; A61K 39/00; A61P 37/06

(52) U.S. Cl. CPC A61K 39/09; A61P 37/06; A61K 2039/55

(57) ABSTRACT

Among the various aspects of the present disclosure is the provision of bacteria compositions and methods of use thereof. Disclosed herein is a bacteria composition comprising a strain of bacteria that translocates from the gut and disseminates to extraintestinal tissue. A method to protect, prevent, and/or treat sepsis that includes administering a bacteria composition to a subject in need is also disclosed. A method to improve microbiota and/or health that includes administering a bacteria composition to a subject in need is also disclosed.

### CROSS-REFERENCE TO RELATED APPLICATIONS

This application claims the benefit under 35 U.S.C. § 119(e) of U.S. Provisional Patent Application No. 63/758,329, filed Feb. 13, 2025, the entire disclosure of which is incorporated herein by reference.

### STATEMENT REGARDING FEDERALLY SPONSORED RESEARCH OR DEVELOPMENT

This invention was made with government support under DK097317 and AI173220 awarded by the National Institutes of Health. The government has certain rights in the invention.

### REFERENCE TO SEQUENCE LISTING

This application includes a Sequence Listing filed electronically as an XML file named 150602117SEQ, created on Feb. 13, 2026, with a size of 3,719 bytes. The Sequence Listing is incorporated herein by reference.

### FIELD OF THE INVENTION

The present disclosure generally relates to methods of protecting a baby from sepsis, comprising administering to the gut of the baby a strain of bacteria that translocates from the gut and disseminates to extraintestinal tissue, wherein the bacteria does not induce a significant inflammatory response or a systemic inflammatory response.

The present disclosure also relates to methods of preventing sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also relates to methods of treating sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also relates to methods of improving the microbiota of a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also relates to methods of improving the health of a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also relates to compositions comprising a strain of bacteria that translocates from the gut and disseminates to extraintestinal tissue, wherein the bacteria does not induce a significant inflammatory response or a systemic inflammatory response, and wherein the composition is formulated for administration to the gut of a baby.

### BACKGROUND OF THE INVENTION

The mammalian gut microbiota harbors vast numbers of diverse microorganisms. The contribution of gut-resident bacteria to host health is well appreciated including supporting gut immune development, protecting from enteric infection and facilitating gut nutrient uptake (Zheng et al., Cell Res, 2020, 30, 492-506; Round et al., Proc Natl Acad Sci USA, 2010, 107, 12204-12209; Verma et al., Sci Immunol, 2018, 3; Jeon et al., PLoS Pathog, 2012, 8, e1002714; Cervantes-Barragan et al., Science, 2017, 357, 806-810; Kim et al., Science, 2017, 356, 315-319; Becattini et al., J Exp Med, 2017, 214, 1973-1989; Faber et al., Nature, 2016, 534, 697-699; and Chen et al., N Engl J Med, 2021, 384, 1517-1528). Some of the reported gut bacteria induced host effects extend to distant tissues, such as the spleen (Rosado et al., Immunol Lett, 2018, 199, 1-15 and Kolypetri et al., iScience, 2021, 24, 102356), thymus (Constantinides et al., Science, 2019, 366 and Zegarra-Ruiz et al., Nature, 2021, 594, 413-417), brain (Morais et al., Nat Rev Microbiol, 2021, 19, 241-255), and mammary glands (Rodriguez, Adv Nutr, 2014, 5, 779-784). Gut-resident bacteria induce these effects on the host via multiple mechanisms such as production of exopolysaccharides (Verma et al., Sci Immunol, 2018, 3 and Mazmanian et al., Cell, 2005, 122, 107-118), bacterial metabolic intermediates (Cervantes-Barragan et al., Science, 2017, 357, 806-810 and Schretter et al., Nature, 2018, 563, 402-406), antimicrobial compounds (Heilbronner et al., Nat Rev Microbiol, 2021, 19, 726-739), short-chain fatty acids (Furusawa et al., Nature, 2013, 504, 446-450 and Smith et al., Science, 2013, 341, 569-573), and by modification of host bile acids (Campbell et al., Nature, 2020, 581, 475-479). Current concepts of how gut-resident bacteria mediate effects at distant sites include local activities of bacterial products that affect circulating cellular populations and/or bacterial products produced in the gut lumen diffusing and acting beyond the gut. While these models are sufficient to explain many of gut-resident bacteria's effects at distant host sites, they appear less adequate to explain distant effects of gut-resident bacteria mediated by products produced in low concentrations and/or distant events mediated by unstable bacterial derived metabolites (Constantinides et al., Science, 2019, 366).

Moreover, some gut-resident bacteria driven host effects are time-limited, only occurring during a period in early-life, and disruption of these critical time-limited events leads to long-term outcomes for the host (Cahenzli et al., Cell host & microbe, 2013, 14, 559-570; Karlsson et al., Eur J Immunol, 1999, 29, 109-118; and Olszak et al., Science, 2012, 336, 489-493). Previous studies have reported that microbial antigen exposure during a specific preweaning period is critical for the induction of immune tolerance to select gut microbes (Knoop et al., Sci Immunol, 2017, 2). Further, DNA of gut-resident bacteria has been found in the spleen and thymus in early life in healthy mice, however, the implications of its presence and how it reaches these distant sites is not understood (Zegarra-Ruiz et al., Nature, 2021, 594, 413-417). The gut microbiota changes dramatically in early life, and it is possible that the transitory presence of select gut microbes explains this restricted interval of benefit conferred by gut-resident microbes. However, the relationship between the presence and/or abundance of these gut resident microbes and their extraintestinal effects is not straightforward as, at least for some microbe dependent events, the specific taxa are more abundant later in life when these events do not occur (Knoop et al., Sci Immunol, 2017, 2). Together these observations could suggest another dimension to interactions with gut microbes that explains why some gut-resident microbe dependent events are limited to early life and occur at extraintestinal sites despite being driven by scarce and/or unstable microbial products.

The healthy gut has barriers preventing translocation and systemic dissemination of live gut-resident bacteria (Doran et al., Cold Spring Harb Perspect Med, 2013, 3; Knoop et al., Mucosal Immunol, 2015, 8, 198-210; Balmer et al., Science translational medicine, 2014, 6, 237ra266; Spadoni et al., Science, 2015, 350, 830-834; and Vaishnava et al., Proc Natl Acad Sci USA, 2008, 105, 20858-20863), an event overwhelmingly viewed as pathologic and detrimental to the host and the microbe. Accordingly, translocation and dissemination of live bacteria as a potential mechanism to promote beneficial gut microbiota mediated events is underexplored. However, in early life the mammalian gut is less developed and viewed as more permeable. The preweaning gut epithelia have differences from the adult including the differential expression of microbial pattern recognition receptors (Fulde et al., Nature, 2018, 560, 489-493 and Pott et al., PLOS Pathog, 2012, 8, e1002670), the presence of vacuolated fetal enterocytes in the small intestine (Trahair et al., J Anat, 1989, 166, 103-111 and Trahair et al., J Pediatr Gastroenterol Nutr, 1995, 21, 277-287), and the presence of physiologically formed colonic goblet cell associated antigen passages (GAPs) (Knoop et al., Sci Immunol, 2017, 2). The current disclosure questioned whether gut-resident bacteria could take advantage of differences in unique early life gut epithelial biology or other unique features to translocate to extraintestinal tissues and mediate some of these beneficial gut microbial events in early life.

### SUMMARY OF THE INVENTION

Among the various aspects of the present disclosure is the provision of bacteria compositions that translocate from the gut and disseminate to extraintestinal tissue and methods of use thereof.

Briefly, therefore, the present disclosure is directed to bacteria compositions that translocate from the gut and disseminate to extraintestinal tissue, wherein the bacteria does not induce a significant inflammatory response or a systemic inflammatory response for use in the treatment and/or prevention of sepsis. In addition, the present disclosure is directed to bacteria compositions that translocate from the gut and disseminate to extraintestinal tissue, wherein the bacteria does not induce a significant inflammatory response or a systemic inflammatory response for use in the improvement of the microbiota and/or health of a baby.

The present teachings also include compositions comprising a strain of bacteria that translocates from the gut and disseminates to extraintestinal tissue.

The present teachings also include compositions comprising a strain of bacteria that translocates from the gut and disseminates to extraintestinal tissue, wherein the bacteria does not induce a significant inflammatory response or a systemic inflammatory response.

The present teachings also include compositions comprising a strain of bacteria that translocates from the gut and disseminates to extraintestinal tissue, wherein the bacteria does not induce a significant inflammatory response or a systemic inflammatory response, and wherein the composition is formulated for administration to the gut of a baby.

The present teachings also include methods of protecting a baby from sepsis, comprising administering to the gut of the baby a strain of bacteria that translocates from the gut and disseminates to extraintestinal tissue.

The present teachings also include methods of protecting a baby from sepsis, comprising administering to the gut of the baby a strain of bacteria that translocates from the gut and disseminates to extraintestinal tissue, wherein the bacteria does not induce a significant inflammatory response or a systemic inflammatory response.

The present teachings also include methods of preventing sepsis in a baby, comprising administering to the gut of the baby a strain of bacteria that translocates from the gut and disseminates to extraintestinal tissue.

The present teachings also include methods of preventing sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present teachings also include methods of treating sepsis in a baby, comprising administering to the gut of the baby a strain of bacteria that translocates from the gut and disseminates to extraintestinal tissue.

The present teachings also include methods of treating sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present teachings also include methods of improving the microbiota of a baby, comprising administering to the gut of the baby a strain of bacteria that translocates from the gut and disseminates to extraintestinal tissue.

The present teachings also include methods of improving the microbiota of a baby, comprising administering to the gut of the baby a strain of bacteria that translocates from the gut and disseminates to extraintestinal tissue, wherein the bacteria does not induce a significant inflammatory response or a systemic inflammatory response.

The present teachings also include methods of improving the health of a baby, comprising administering to the gut of the baby a strain of bacteria that translocates from the gut and disseminates to extraintestinal tissue.

The present teachings also include methods of improving the health of a baby, comprising administering to the gut of the baby a strain of bacteria that translocates from the gut and disseminates to extraintestinal tissue, wherein the bacteria does not induce a significant inflammatory response or a systemic inflammatory response.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A illustrates a schematic representation of the experimental set up used in the identification of translocating bacteria in mice.

FIG. 1B illustrates CFU/organ (colony forming units per organ) of bacteria recovered from brain heart infusion (BHI) plates of spleen and MLN homogenates from DOL17 and DOL35 littermates (n=27 for DOL17 MLN, n=26 for DOL35 MLN, n=15 for DOL17 spleen and n=14 for DOL35 spleen). Litters are color-coded, circle denotes females, and triangle denotes males to demonstrate that translocation was not litter or sex dependent but was age dependent. Statistical comparisons were performed using a one-sided cumulative binomial distribution probability and two-tailed Students t-test for intestinal tissues, P values are as denoted.

FIG. 1C illustrates specific bacterial taxa identified from six DOL17 mice (M1-M6) MLNs by full length 16s rRNA Sanger sequencing of isolates.

FIG. 1D illustrates schematic representation of the experimental set up used to track L. animalis^{WU} colonization in intestinal tissues and translocation to MLN and spleen in DOL17 and DOL35 mice.

FIG. 1E illustrates CFU/organ of L. animalis^{WU} recovered from intestinal and extraintestinal tissues of L. animalis^{WU} fed DOL17 (N=15) and DOL35 (N=15) mice. Litters are color-coded, circle denotes females, and triangle denotes males to demonstrate that translocation was not litter or sex dependent but was age dependent. Statistical comparisons were performed using a one-sided cumulative binomial distribution probability two-tailed Students t-test for intestinal tissues, P values are as denoted.

FIG. 1F illustrates pattern of number of CFUs recovered from the MLN and spleen of DOL17 mice.

FIG. 1G illustrates relative frequency of bacterial taxa at family level compared by 16S v4 sequencing of specific pathogen free laboratory mice at DOL17 (n=3) and DOL35 (n=4). Lactobacilluse family (denoted by red box in the legend and arrows on the graph) was one of the most common translocating taxa, is present at both ages.

FIG. 1H illustrates the culture of luminal vs mucosal scrapings from L. animalis^{WU} fed DOL17 mice (n=4) reveals that L. animalis^{WU} can be present in both compartments.

FIG. 1I illustrates a schematic of EGF treatment to inhibit GAPs and FTY720 treatment to inhibit trafficking.

FIG. 1J illustrates assessment of gut barrier function in L. animalis^{WU} colonized mice in the presence and absence of EGF (n=3 per treatment group) using the 4 kD FITC dextran leak assay. Graphs represent the mean±/−SEM. P value calculated using two-tailed Student's t-test.

FIG. 2A illustrates representative images from the proximal colon of DOL17 mice with gut-resident bacteria (eubacterial FISH probe; red) identified within a goblet cell (UEA1+ green) and DAPI (in blue) visualized in XY, YZ and XZ plane and is representative of colon sections from unmanipulated DOL17 mice from 3 independent litters in which 13 of 125 goblet cells (UEA1+) and 1 of 553 (UEA1−) colonocytes imaged in 3 dimensions (z-stacks) contained bacteria (eubacterial FISH probe+).

FIG. 2B illustrates CFU/organ of L. animalis^{WU} recovered from intestinal and extraintestinal tissues of Math1^{f/f} (n=9) and goblet cell deficient Math1^{f/f}vil-Cre-ER^{T2} (n=13) DOL17 mice fed with L. animalis^{WU}. A two-tailed Student's t-test and a one-sided cumulative binomial distribution probability test were used for extraintestinal tissues used.

FIG. 2C illustrates CFU/organ of L. animalis^{WU} recovered from the intestine and extraintestinal tissues of nontreated (n=14), EGF treated (n=14) and pan-S1PR modulator (FTY720) treated (n=6) DOL17 mice fed with L. animalis^{WU}. A one-sided cumulative binomial distribution probability test was used for extraintestinal tissues. In addition, a one-way ANOVA with at Dunnett's post test was used for intestinal tissues.

FIG. 2D illustrates flow cytometry plots showing depletion of F4/80+ MHCII+ CD45+ splenic cells in Clodrosome treated preweaning mice (n=5) when compared to nontreated mice (n=4).

FIG. 2E illustrates graph showing depletion of F4/80+ MHCII+ CD45+ splenic cells in Clodrosome treated preweaning mice (n=5) when compared to nontreated mice (n=4). A two-tailed Student's t-test was used.

FIG. 2F illustrates CFU/organ of L. animalis^{WU} recovered intestinal and extraintestinal tissues of PBS (n=4) and Clodrosome (n=4) treated preweaning mice. A two-tailed Student's t-test and a one-sided cumulative binomial distribution probability test were used for extraintestinal tissues.

FIG. 2G illustrates CFU of in vitro cultured L. animalis^{WU} recovered after treatment of 103 L. animalis^{WU} with gentamicin at the denoted concentrations for 2 hours. A one-way ANOVA with at Dunnett's post test was used for intestinal tissues.

FIG. 2H illustrates CFU/organ of L. animalis^{WU} recovered from MLNs (n=3) of in vitro cultured L. animalis^{WU} recovered after treatment of 10³ L. animalis^{WU} with gentamicin at the denoted concentrations for 2 hours. A two-tailed Student's t-test was used.

FIG. 2I illustrates CFU/organ of L. animalis^{WU} recovered from spleens (n=4) of in vitro cultured L. animalis^{WU} recovered after treatment of 10³ L. animalis^{WU} with gentamicin at the denoted concentrations for 2 hours. A two-tailed Student's t-test was used.

FIG. 2J illustrates percentage of immune cells expressing S1PR (% S1P^{RGFP+}CD45⁺) assessed in colon and spleen of S1PR-GFP reporter mice (B6.129P2-S1pr1^{tm1Hrose}/J) treated at DOL17 with pan-S1PR inhibitor (FTY720) (n=3) or not treated (control) (n=3). Statistical analyses were performed by two-tailed Student's t test in GraphPad Prism. Data represented as mean with individual values. P values are as denoted.

FIG. 3A illustrates a schematic representation of experimental setup used in investigating transcriptomic changes associated with L. animalis^{WU} translocation in MLN of DOL17 mice.

FIG. 3B illustrates a heatmap of 69 differentially expressed genes in sorted cellular populations defined as in FIG. 3A and confirmed by CFUs in the liver. Statistical analyses performed using DEseq2 with FDR<0.05 and >2 fold change in Partek® FLOW®.

FIG. 3C illustrates peripheral blood neutrophils and serum measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3D illustrates IL6 measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3E illustrates TNFα measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3F illustrates IL10 measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3G illustrates CCL2 measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3H illustrates CXCL9 measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3I illustrates CXCL10 measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3J illustrates IFNα measured in DOL17 mice that are non-treated (n=6) or treated with L. animalis^{WU} fed orally (L. ani oral) (n=6) or administered by i.p. injection (L. ani i.p.) (n=6). Graphs presented as mean±/−SEM. Statistical analyses performed with one-way ANOVA with a Dunnett's post test.

FIG. 3K illustrates flow cytometry plots demonstrating CSF1R blockade, loss of cDC1 cells, and CX₃CR1 deletion does not impair L. animalis^{WU} translocation in preweaning mice.

FIG. 3L illustrates CSFR1 blockade in preweaning mice (n=3) reduces the CD45+ colonic LP cellular population which can also express CX₃CR1 and F4/80. Graphs represent mean±/−SEM. Statistical analyses were performed by two-tailed Student's t test.

FIG. 3M illustrates CSFR1 blockade in preweaning mice (n=3) reduces the MHCII+ colonic LP cellular population which can also express CX₃CR1 and F4/80. Graphs represent mean±/−SEM. Statistical analyses were performed by two-tailed Student's t test.

FIG. 3N illustrates CSFR1 blockade in preweaning mice (n=3) reduces the MHCII+ CD11c− colonic LP cellular population which can also express CX₃CR1 and F4/80. Graphs represent mean±/−SEM. Statistical analyses were performed by two-tailed Student's t test.

FIG. 3O illustrates CSFR1 blockade in preweaning mice (n=3) reduces the MHCII+ CD11c− colonic LP cellular population which can also express CX₃CR1 and F4/80. Graphs represent mean±/−SEM. Statistical analyses were performed by two-tailed Student's t test.

FIG. 3P illustrates CFU/organ of L. animalis^{WU} recovered from intestinal and extraintestinal tissues of L. animalis^{WU} fed preweaning mice that were nontreated controls (n=4) or treated with anti-CSF1R (n=3). Graphs represent mean±/−SEM. Statistical analyses were performed by two-tailed Student's t test and one-sided cumulative binomial distribution probability test for extraintestinal tissues.

FIG. 3Q illustrates CFU/organ of L. animalis^{WU} recovered from intestinal and extraintestinal tissues of L. animalis^{WU} fed wildtype (n=6), cDC1 deficient (Irf8 delta 32) mice (n=6), or CX3CR1 deficient preweaning mice (n=5). Graphs represent mean±/−SEM. Statistical analyses were performed by one-way ANOVA and one-sided cumulative binomial distribution probability test for extraintestinal tissues for intestinal tissues.

FIG. 4A illustrates putative gramicidin-tyrocidine antibiotic coding sequences identified in L. animalis^{WU} by Prokka annotation and five other gene regions predicted by antiSMASH to encode secondary metabolites with potential antimicrobial activity (red).

FIG. 4B illustrates representative images of antibacterial activity of L. animalis^{WU} by agar diffusion in E. coli ST69 LB plates incubated with MRS broth (control), supernatant from overnight L. animalis^{WU} culture, L. animalis^{WU} culture supernatant treated with proteinase K (experiment was performed three times).

FIG. 4C illustrates E. coli ST69 recovery from 6 hour cultured E. coli ST69 (n=3 per treatment group) grown in LB broth with no additional treatments (growth control), treated with MRS broth (vehicle control), L. animalis^{WU} culture supernatant or, L. animalis^{WU} culture supernatant treated with proteinase K, plated on LB agar. Statistical analyses were performed by one-way ANOVA with a Dunnett's post test.

FIG. 4D illustrates a schematic representation of an experimental setup used to assess the protective role of bacterial translocation in preweaning mice against systemic E. coli ST69 infection.

FIG. 4E illustrates a comparison of survival in nontreated (n=9) and VNAM antibiotic treated (n=7) mice. Statistical analyses were performed by Log Rank (Mantel-Cox) test in GraphPad Prism.

FIG. 4F illustrates L. animalis^{WU} (n=5) and EGF+L. animalis^{WU} treated (n=5) mice that were infected with E. coli ST69 by i.p. injection. Statistical analyses were performed by Log Rank (Mantel-Cox) test in GraphPad Prism.

FIG. 4G illustrates body weight of segmented neutrophils in the peripheral blood. Statistical analyses were performed by a two-tailed Student's t test.

FIG. 4H illustrates percentage of segmented neutrophils in the peripheral blood. Statistical analyses were performed by a two-tailed Student's t test.

FIG. 4I illustrates representative images of hepatic abscess (marked by black arrows in mice in both groups).

FIG. 4J illustrates quantification of hepatic abscesses per mm² in L. animalis^{WU} (n=4) and EGF+L. animalis^{WU} treated (n=4) mice infected 2.5 days post infection with E. coli ST69 by i.p. injection. Statistical analyses were performed by a two-tailed Student's t test.

FIG. 4K illustrates flow cytometry gating strategy for identifying Th1 cytokines (TNFα and IFNγ) secreted by CD45⁺CD3⁺CD4⁺ T cells in MLNs and spleens.

FIG. 4L illustrates frequency of TNFα+ and IFNγ+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4M illustrates frequency of TNFα+ and IFNγ+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4N illustrates frequency of TNFα+ and IFNγ+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4O illustrates frequency of TNFα+ and IFNγ+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4P illustrates flow cytometry gating strategy for identifying Th2 cytokines (IL4 and IL13) secreted by CD45⁺CD3⁺CD4⁺ T cells in MLNs and spleens.

FIG. 4Q illustrates frequency of IL13+ and IL4+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4R illustrates frequency of IL13+ and IL4+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4S illustrates frequency of IL13+ and IL4+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4T illustrates frequency of IL13+ and IL4+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4U illustrates flow cytometry gating strategy for identifying Th17 cytokines (IL17 and IL22) secreted by CD45⁺CD3⁺CD4⁺ T cells in MLNs and spleens.

FIG. 4V illustrates frequency of IL17+ and IL22+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4W illustrates frequency of IL17+ and IL22+ CD4+ T cells in MLNs of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4X illustrates frequency of IL17+ and IL22+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 4Y illustrates frequency of IL17+ and IL22+ CD4+ T cells in spleens of nontreated (control) (n=9), L. animalis^{WU} fed (n=5) and L. animalis^{WU}+EGF fed mice (n=6). Statistical analyses were performed by one-way ANOVA with Dunnett's post test. Graphs represent mean±/−SEM. P values are as denoted.

FIG. 5 illustrates neutrophil (CD45⁺Ly6G/C⁺ cells) numbers in non-infected mice (n=3) or mice infected with E. coli ST69 (n=5) gavaged orally at DOL17. Statistical analyses were performed by two-tailed Student's t test in GraphPad Prism. Graph represents mean±/−SEM. P values are as denoted.

FIG. 6A illustrates graphs of Foxp3+ and RORγt+ Foxp3+ regulatory T cell populations in the MLN and spleen of unmanipulated mice (Control) (n=4 for MLNs and 3 for spleens), mice fed L. animalis^{WU} from DOL10-20 (L. animalis^{WU}) (n=4 for MLNs and 3 for spleens), and mice fed L. animalis^{WU} given EGF gavage from DOL10-20 (L. animalis^{WU}+EGF) (n=4 for MLNs and spleen). Graphs represent mean±/−SEM. Statistical analyses were performed by one way ANOVA with a Dunnett's post test.

FIG. 6B illustrates representative flow plots of Foxp3+ and RORγt+ Foxp3+ regulatory T cell populations in the MLN and spleen of unmanipulated mice (Control) (n=4 for MLNs and 3 for spleens), mice fed L. animalis^{WU} from DOL10-20 (L. animalis^{WU}) (n=4 for MLNs and 3 for spleens), and mice fed L. animalis^{WU} given EGF gavage from DOL10-20 (L. animalis^{WU}+EGF) (n=4 for MLNs and spleen).

FIG. 7A illustrates cellular populations from the MLN isolated and treated with propidium monoazide and photoactivation, DNA was isolated and bacterial taxa were characterized by 16s rRNA v4 sequencing. The number of different taxa identified were not dramatically altered by L. animalis^{WU} feeding.

FIG. 7B illustrates cellular populations from the spleen isolated and treated with propidium monoazide and photoactivation, DNA was isolated and bacterial taxa were characterized by 16s rRNA v4 sequencing. The number of different taxa identified were not dramatically altered by L. animalis^{WU} feeding.

FIG. 8A illustrates colony forming units (CFU) of E. coli ST69 (n=3) after plating bacteria treated with Vancomycin at the specified concentrations for 4 hours. Statistical analyses were performed by one way ANOVA with a Dunnett's post test. Data represented as mean±/−SEM P values are as denoted.

FIG. 8B illustrates colony forming units (CFU) of E. coli ST69 (n=3) after plating bacteria treated with Neomycin at the specified concentrations for 4 hours. Statistical analyses were performed by one way ANOVA with a Dunnett's post test. Data represented as mean±/−SEM P values are as denoted.

FIG. 8C illustrates colony forming units (CFU) of E. coli ST69 (n=3) after plating bacteria treated with Ampicillin at the specified concentrations for 4 hours. Statistical analyses were performed by one way ANOVA with a Dunnett's post test. Data represented as mean±/−SEM P values are as denoted.

FIG. 8D illustrates colony forming units (CFU) of E. coli ST69 (n=3) after plating bacteria treated with Metronidazole at the specified concentrations for 4 hours. Statistical analyses were performed by one way ANOVA with a Dunnett's post test. Data represented as mean±/−SEM P values are as denoted.

FIG. 8E illustrates colony forming units (CFU) of L. animalis^{WU} (n=3) after plating bacteria treated with Vancomycin at the specified concentrations for 4 hours. Statistical analyses were performed by one way ANOVA with a Dunnett's post test. Data represented as mean±/−SEM P values are as denoted.

FIG. 8F illustrates colony forming units (CFU) of L. animalis^{WU} (n=3) after plating bacteria treated with Neomycin at the specified concentrations for 4 hours. Statistical analyses were performed by one way ANOVA with a Dunnett's post test. Data represented as mean±/−SEM P values are as denoted.

FIG. 8G illustrates colony forming units (CFU) of L. animalis^{WU} (n=3) after plating bacteria treated with Ampicillin at the specified concentrations for 4 hours. Statistical analyses were performed by one way ANOVA with a Dunnett's post test. Data represented as mean±/−SEM P values are as denoted.

FIG. 8H illustrates colony forming units (CFU) of L. animalis^{WU} (n=3) after plating bacteria treated with Metronidazole at the specified concentrations for 4 hours. Statistical analyses were performed by one way ANOVA with a Dunnett's post test. Data represented as mean±/−SEM P values are as denoted.

### DETAILED DESCRIPTION OF THE INVENTION

The present disclosure is based, at least in part, on the discovery that bacteria compositions that translocate from the gut and disseminate to extraintestinal tissue can be used for the treatment/prevention/protection of sepsis and/or improve the microbiota/health of a baby.

One aspect of the present disclosure provides for compositions comprising a strain of bacteria that translocates from the gut and disseminates to extraintestinal tissue.

The present disclosure also includes compositions comprising a strain of bacteria that translocates from the gut and disseminates to extraintestinal tissue, wherein the bacteria does not induce a significant inflammatory response or a systemic inflammatory response.

The present disclosure also includes compositions comprising a strain of bacteria that translocates from the gut and disseminates to extraintestinal tissue, wherein the bacteria does not induce a significant inflammatory response or a systemic inflammatory response, and wherein the composition is formulated for administration to the gut of a baby.

The present disclosure also includes methods of protecting a baby from sepsis, comprising administering to the gut of the baby a strain of bacteria that translocates from the gut and disseminates to extraintestinal tissue.

The present disclosure also includes methods of protecting a baby from sepsis, comprising administering to the gut of the baby a strain of bacteria that translocates from the gut and disseminates to extraintestinal tissue, wherein the bacteria does not induce a significant inflammatory response or a systemic inflammatory response.

The present disclosure also includes methods of preventing sepsis in a baby, comprising administering to the gut of the baby a strain of bacteria that translocates from the gut and disseminates to extraintestinal tissue.

The present disclosure also includes methods of preventing sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also includes methods of treating sepsis in a baby, comprising administering to the gut of the baby a strain of bacteria that translocates from the gut and disseminates to extraintestinal tissue.

The present disclosure also includes methods of treating sepsis in a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also includes methods of improving the microbiota of a baby, comprising administering to the gut of the baby a strain of bacteria that translocates from the gut and disseminates to extraintestinal tissue.

The present disclosure also includes methods of improving the microbiota of a baby, comprising administering to the gut of the baby a strain of bacteria that translocates from the gut and disseminates to extraintestinal tissue, wherein the bacteria does not induce a significant inflammatory response or a systemic inflammatory response.

The present disclosure also includes methods of improving the health of a baby, comprising administering to the gut of the baby a strain of bacteria that translocates from the gut and disseminates to extraintestinal tissue.

The present disclosure also includes methods of improving the health of a baby, comprising administering to the gut of the baby a strain of bacteria that translocates from the gut and disseminates to extraintestinal tissue, wherein the bacteria does not induce a significant inflammatory response or a systemic inflammatory response.

In one aspect, any of the methods comprise administering a composition comprising a strain of bacteria that translocates from the gut and disseminates to extraintestinal tissue.

In another aspect, any of the methods comprise administering a composition comprising a strain of bacteria that translocates from the gut and disseminates to extraintestinal tissue, wherein the bacteria does not induce a significant inflammatory response or a systemic inflammatory response.

In another aspect, any of the methods comprise administering a composition comprising a strain of bacteria that translocates from the gut and disseminates to extraintestinal tissue, wherein the bacteria does not induce a significant inflammatory response or a systemic inflammatory response, and wherein the composition is formulated for administration to the gut of a baby.

In another aspect, in any of the methods the baby is a newborn. In another aspect, in any of the methods the baby is up to 2 or 3 months old. In another aspect, the baby is up to 6 months old. In another aspect, in any of the methods administration of the bacteria to the gut comprises oral administration. In another aspect, in any of the methods translocation from the gut comprises Goblet cell-mediated translocation. In another aspect, in any of the methods the bacteria comprises a strain of Lactobacillus. In another aspect, in any of the methods the bacteria comprises Lactobacillus animalis. In another aspect, in any of the methods the strain of bacteria comprises Lactobacillus animalis^{WU}.

In another aspect, the composition can include a foodstuff. In another aspect, the composition can include a buffer, a salt, or a sugar. In another aspect, the composition is administered orally. In another aspect, the bacteria is lyophilized (freeze-dried). In another aspect, translocation of the bacteria comprises Goblet cell-mediate translocation. In another aspect, the bacteria comprises a strain of Lactobacillus. In another aspect, the strain of bacteria comprises Lactobacillus animalis. In another aspect, the strain of bacteria comprises Lactobacillus animalis^{WU}.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or protecting from sepsis in a subject in need of administration of a therapeutically effective amount of a bacteria composition and any combination thereof, so as to alleviate sepsis.

Methods described herein are generally performed on a subject in need thereof. A subject in need of the therapeutic methods described herein can be a subject having, diagnosed with, suspected of having, or at risk for developing sepsis. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a bacteria composition is, for example, an amount that would cause the desired therapeutic effect in a subject while minimizing undesired side effects. In various embodiments, an effective amount of a bacteria composition described herein can substantially inhibit sepsis, slow the progress of sepsis, or limit the development of sepsis.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a bacteria composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat sepsis.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a bacteria composition can occur as a single event or over a time course of treatment. For example, a bacteria composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for sepsis.

A bacteria composition can be administered simultaneously or sequentially with another agent, such as an antibiotic, an anti-inflammatory, or another agent. For example, a bacteria composition can be administered simultaneously with another agent, such as an antibiotic or an anti-inflammatory. Simultaneous administration can occur through the administration of separate compositions, each containing one or more of a bacteria composition, an antibiotic, an anti-inflammatory, or another agent. Simultaneous administration can occur through the administration of one composition containing two or more of a bacteria composition, an antibiotic, an anti-inflammatory, or another agent. A bacteria composition can be administered sequentially with an antibiotic, an anti-inflammatory, or another agent. For example, a bacteria composition can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Select Gut Bacteria Translocate and Disseminate in Early Life

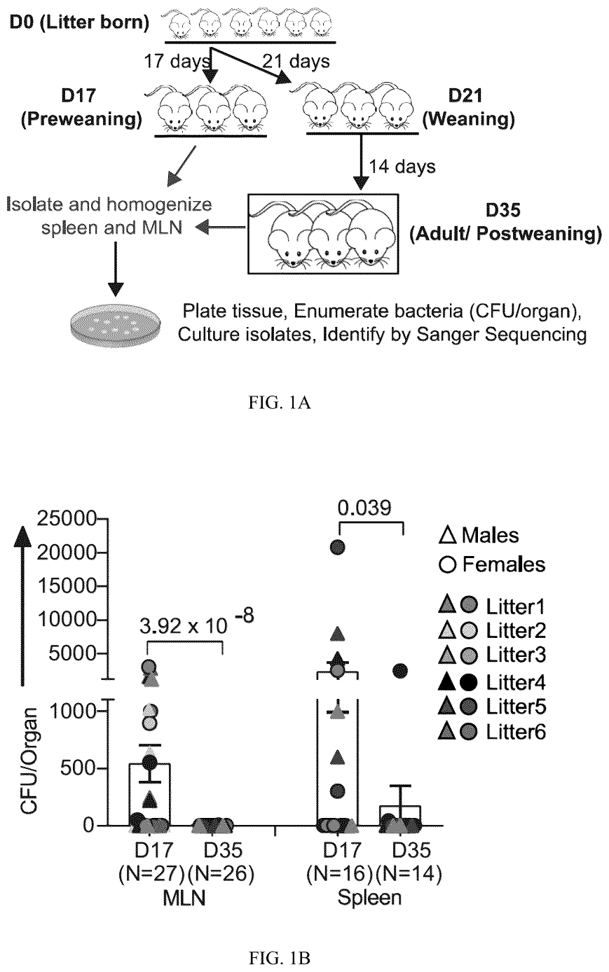

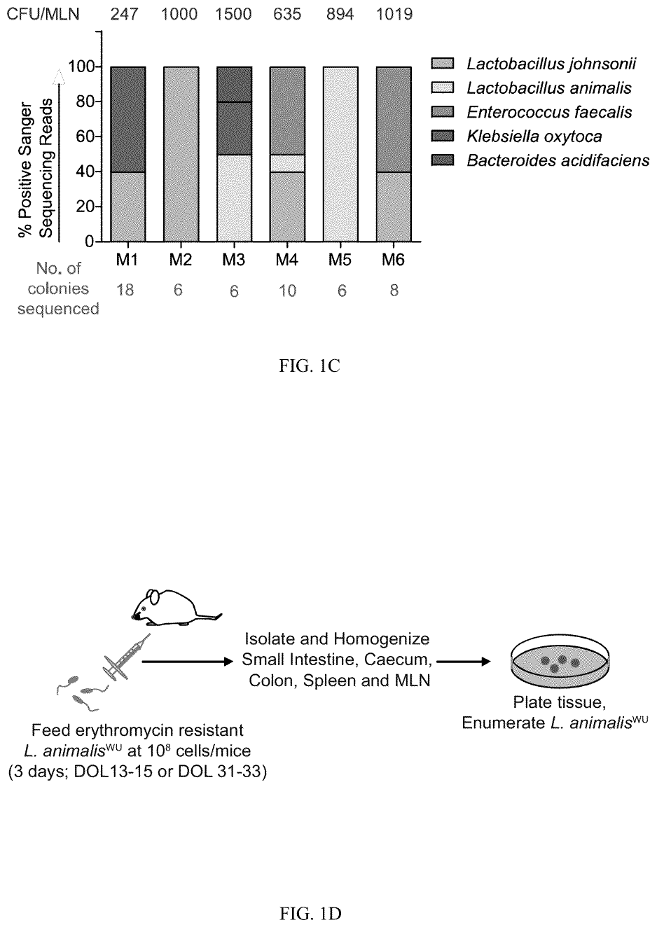

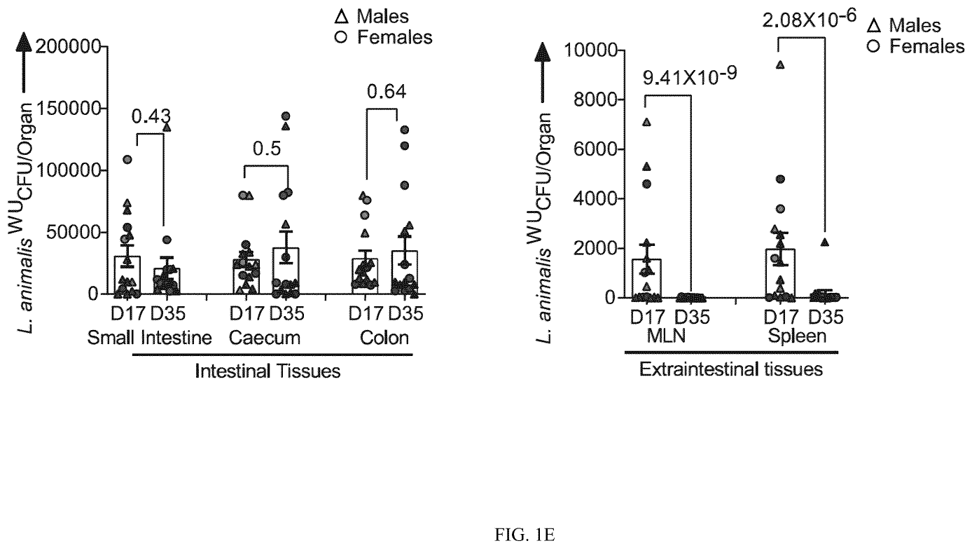

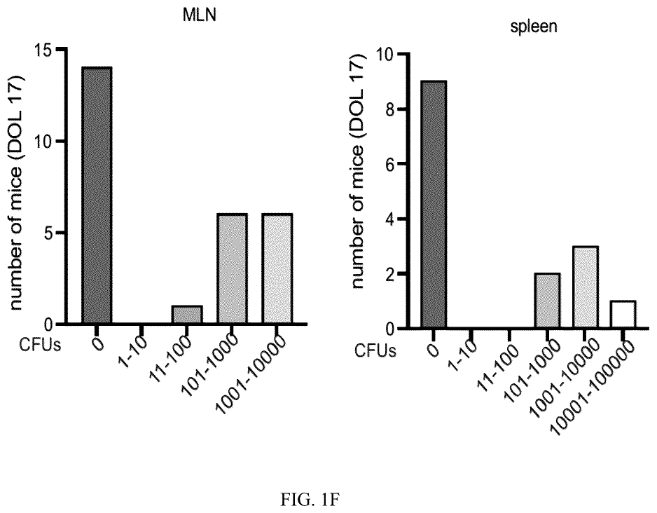

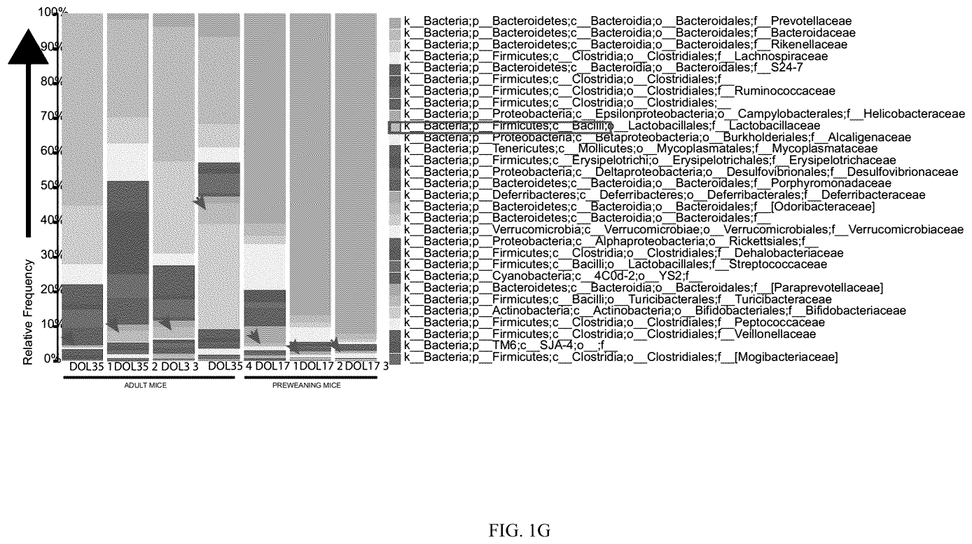

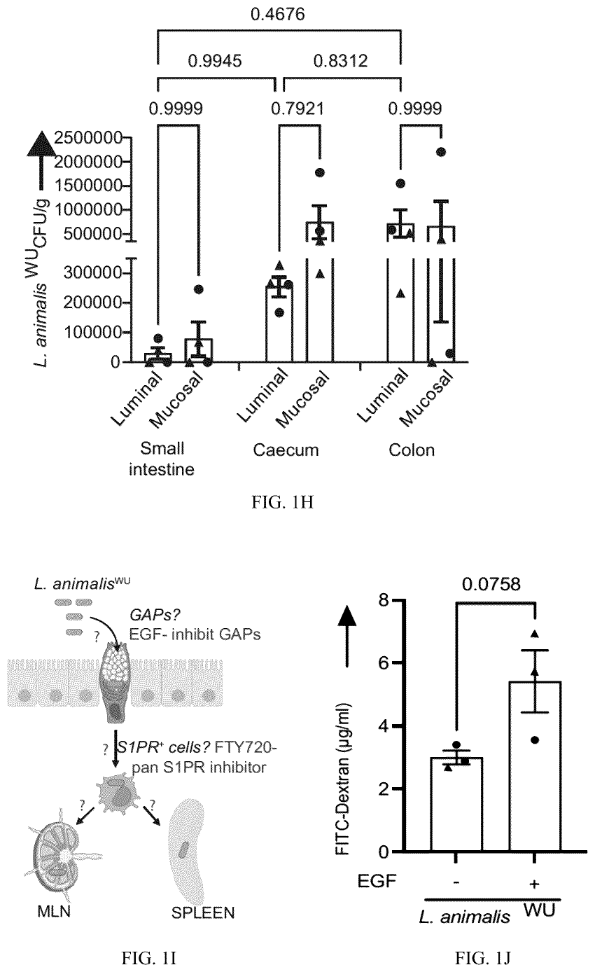

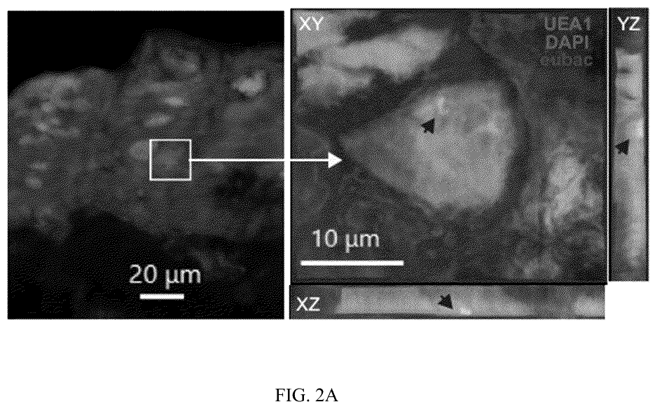

Day of life 17 (DOL17; preweaning) mice, a time of life in which there is altered intestinal epithelial cell TLRs expression (Knoop et al., Sci Immunol, 2017, 2), the presence of vacuolated enterocytes in rodents (Trahair et al., J Pediatr Gastroenterol Nutr, 1995, 21, 277-287), and the presence of physiologically formed colonic GAPs (Knoop et al., Sci Immunol, 2017, 2), were evaluated for translocation and dissemination of gut-resident bacteria. Multiple bacterial culture media/conditions were tested and it was found that culture with brain heart infusion (BHI) media recovered all live bacterial taxa, identified by full length 16s rRNA sequencing of isolates, recovered from the mesenteric lymph nodes (MLNs) of DOL17 SPF housed C57BL/6 mice. Interestingly there was no observation of live bacteria in MLNs of SPF housed C57BL/6 DOL35 (adult) mice in any culture condition. Accordingly, this approach was employed to evaluate translocation in greater depth. To mitigate litter effects and evaluate effects of sex, preweaning (DOL17) mice and adult (DOL35) mice from the same litter were evaluated and analyzed by sex (FIG. 1A). Again, it was observed that live bacteria were recovered significantly more often from the extraintestinal tissues (MLNs and spleen) of preweaning mice when compared to adult mice and that this phenomenon was not related to litter or sex, but highly dependent upon age (FIG. 1B). Translocation of bacteria in preweaning mice was seen in a binomial distribution, with hundreds-thousands of organisms detected in a tissue or not occurring at all within the same litter (FIG. 1B and FIG. 1F). Mice with translocation appeared healthy and were indistinguishable from their littermates in which translocation was not detected. Using full-length 16s rRNA sequencing a limited number of bacterial taxa were observed, dominated by Lactobacillus species, in the MLNs of preweaning mice, which represented a minor subset of the taxa present within the gut lumen (FIG. 1C and FIG. 1G). Thus, live bacteria spontaneously translocated in preweaning mice under physiological conditions and this stochastic event depended upon age but not sex or litter.

The gut microbial community differs dramatically between preweaning and postweaning mice and therefore it was questioned if the lack of translocation in postweaning mice was due to the lack of specific bacterial strains with the property to translocate. An antibiotic-resistant translocating Lactobacillus animalis strain isolated from the MLN of DOL17 mice (hereafter referred to as L. animalis^{WU}) was generated and mice were gavaged with this isolate (FIG. 1D). L. animalis^{WU} colonized the intestinal tract of DOL17 and DOL35 mice equivalently and was found in both the luminal contents and mucosal scraping, suggesting it can reside in the lumen and close to the epithelium (FIG. 1H), but translocated significantly more often in DOL17 mice (FIG. 1E). All preweaning mice colonized with L. animalis^{WU} appeared healthy irrespective of translocation. Translocation of L. animalis^{WU} in preweaning mice again appeared to be stochastic with a binomial distribution despite high levels of L. animalis^{WU} colonization of the intestinal tract (FIG. 1E), indicating that factors other than the presence/abundance of bacteria with the ability to translocate were driving this process. Therefore, further experiments were explored to understand host dependent mechanisms facilitating translocation of L. animalis^{WU} in early life.

### Example 2—Early Life Translocation is Facilitated by the Host

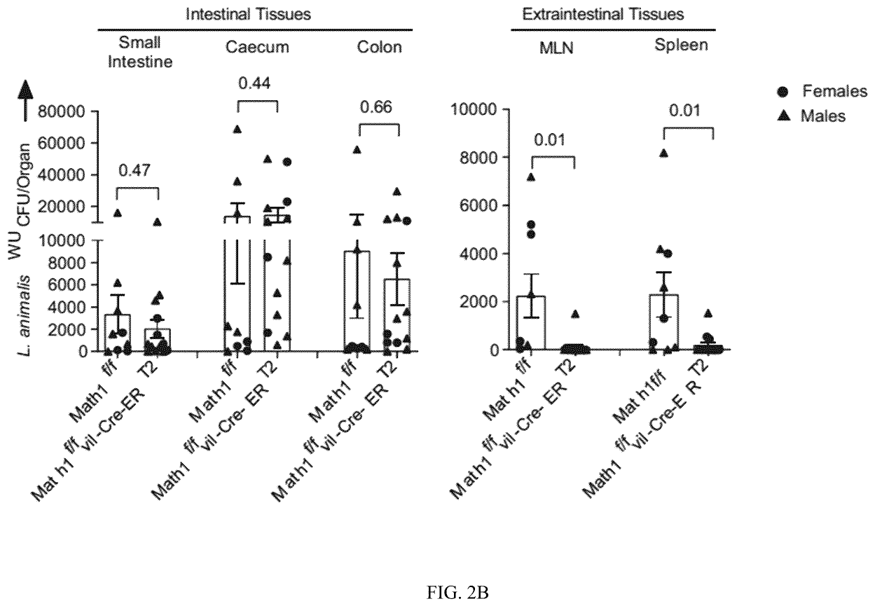

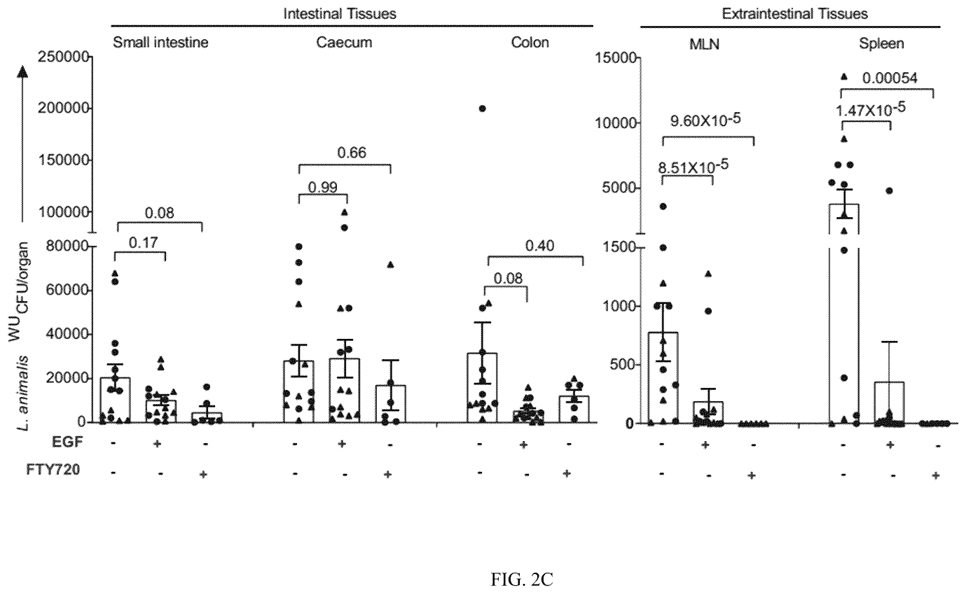

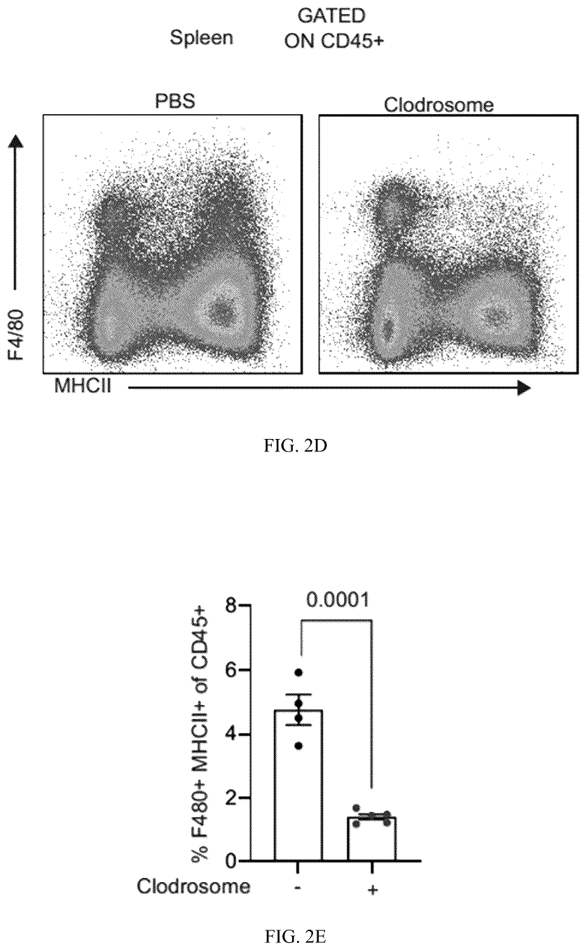

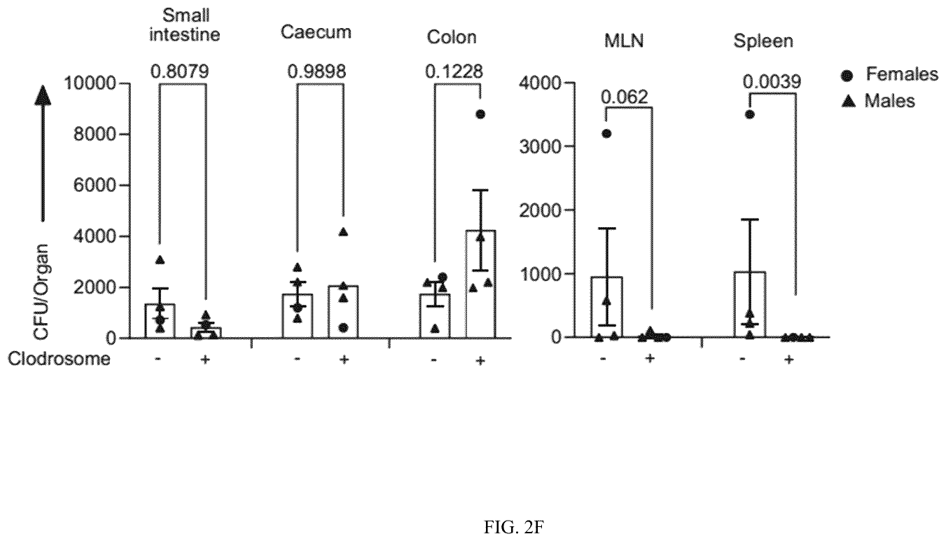

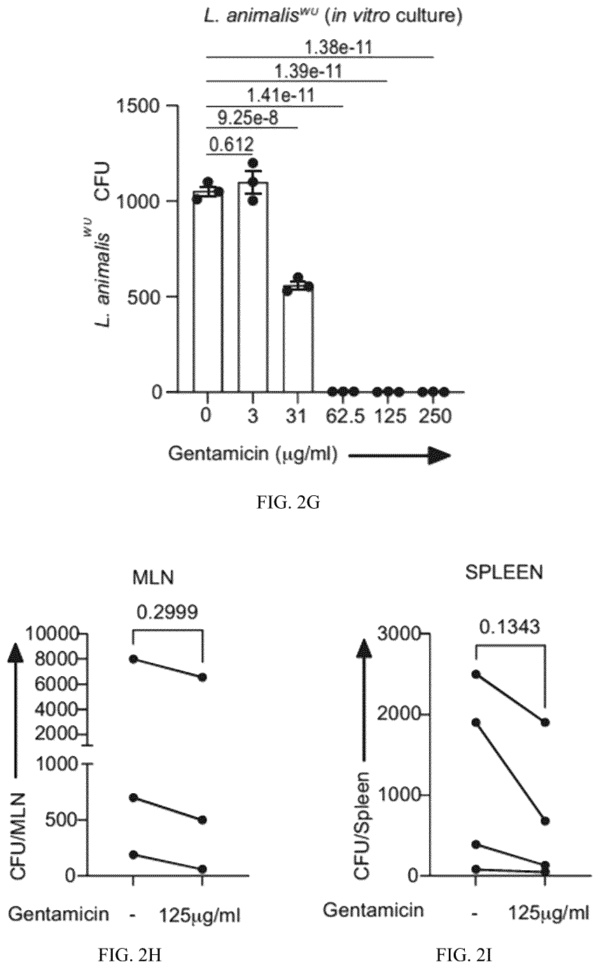

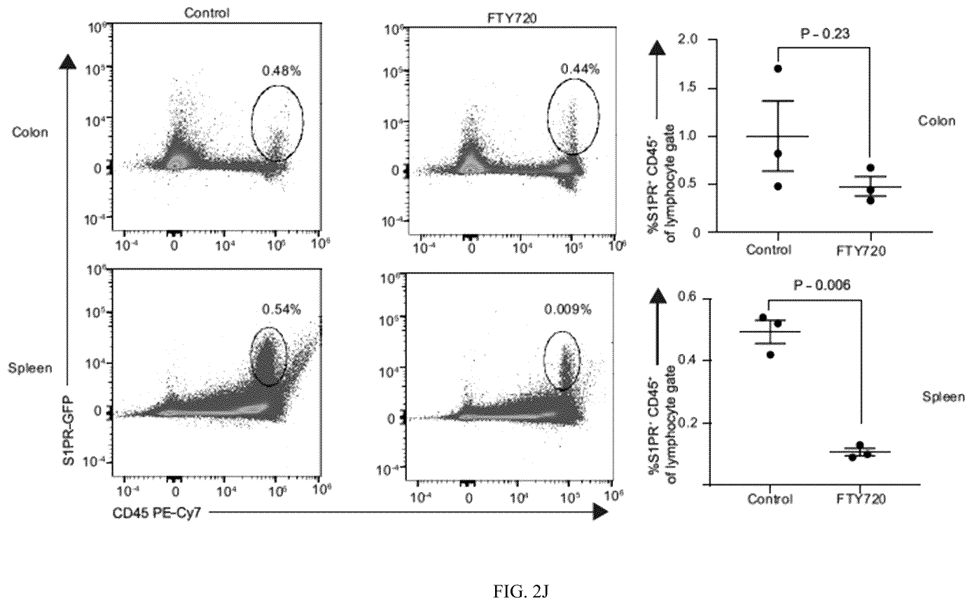

Intestinal goblet cells (GCs) can form GC associated antigen passages (GAPs) to deliver luminal substances to the lamina propria immune system (McDole et al., Nature, 2012, 483, 345-349). In adult mice, GAPs are rare in the colon in the healthy state due to GC intrinsic Myd88 dependent sensing of the gut microbiota, suppressing colonic GCs' ability to form GAPs (Knoop et al., Mucosal Immunol, 2015, 8, 198-210). However, when colonic GAPs form pathologically in adult mice they facilitate the translocation of gut-resident bacteria and induce inflammatory responses (Knoop et al., Gut, 2016, 65, 1100-1109). In contrast colonic GAPs physiologically form for a defined preweaning period in early life which overlaps with the timing of translocation (Knoop et al., Sci Immunol, 2017, 2). Therefore, experiments were designed to evaluate if translocation was dependent upon GCs and GAPs. Gut bacteria could be found within colonic GCs of unmanipulated DOL17 mice using an eubacterial fluorescence in situ hybridization (FISH) probe (FIG. 2A) consistent with GCs/GAPs facilitating gut-resident bacterial translocation. Mouse atonal homologue 1 (Math1) is a transcription factor required for intestinal GC development. Deletion of GCs in preweaning mice through inducible deletion of Math1 in epithelial cells (Math1^{f/f} Vil-Cre-ER^{T2} mice) did not impair the ability of L. animalis^{WU} to colonize the intestinal tract but significantly impaired the translocation and dissemination of L. animalis^{WU} (FIG. 2B). Notably deletion of GCs increases intestinal leak (Kulkarni et al., Mucosal Immunol, 2019) while paradoxically inhibiting translocation, consistent with translocation being a controlled rather than sporadic event. Prior to DOL10, colonic GAPs are inhibited by high luminal levels of epidermal growth factor (EGF) originating from the breastmilk, and gavage with exogenous EGF after DOL10 inhibits colonic GAPs (Knoop et al., Proc Natl Acad Sci USA, 2020, 117, 7941-7949). Gavaging preweaning mice with EGF to inhibit GAPs (FIG. 1I) did not increase gut barrier function (FIG. 1J), and trended toward increasing leak, or impair the ability of L. animalis^{WU} to colonize the intestinal tract, but significantly impaired the ability of L. animalis^{WU} to translocate to the MLN and spleen (FIG. 2D), consistent with the role of GAPs facilitating translocation in preweaning mice.

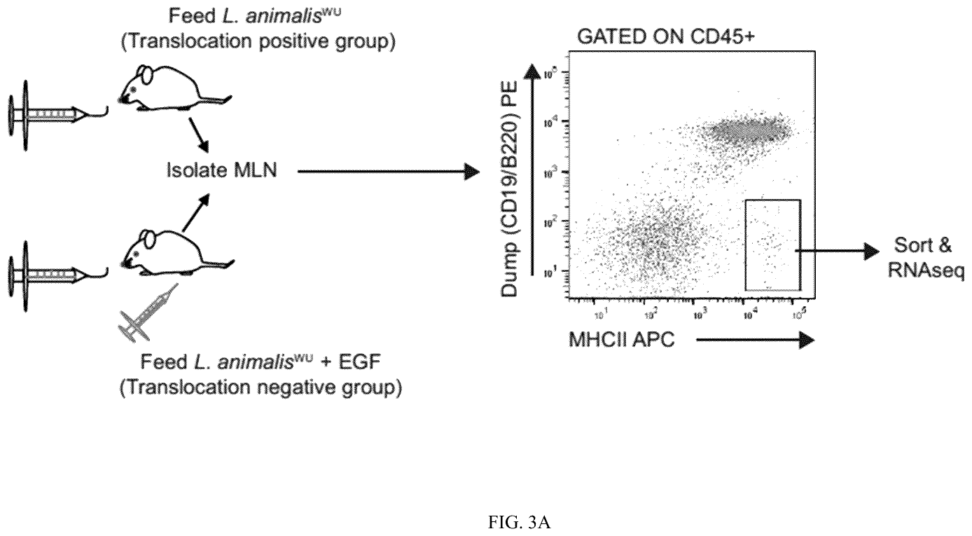

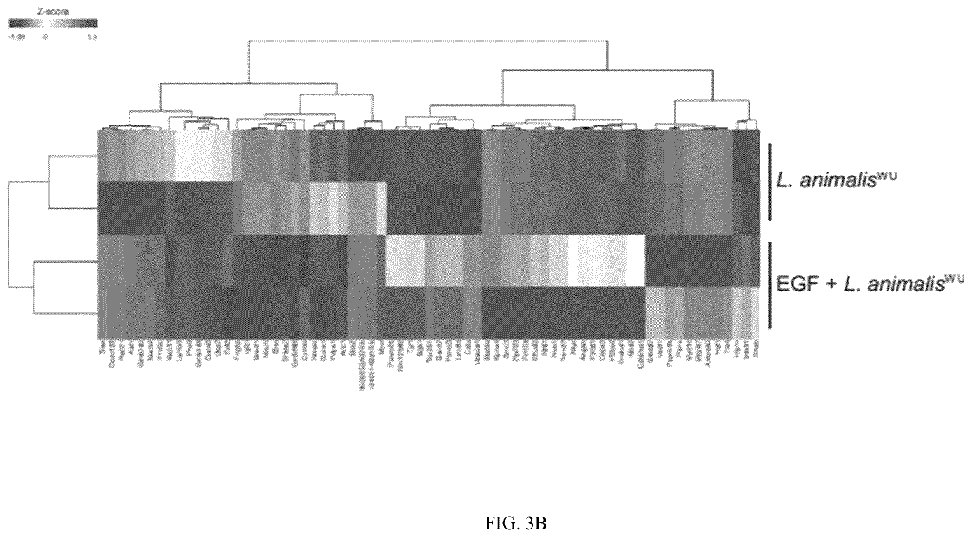

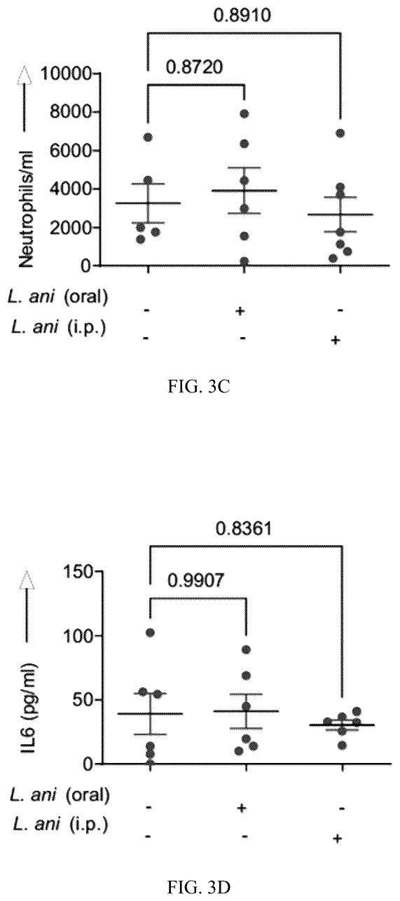

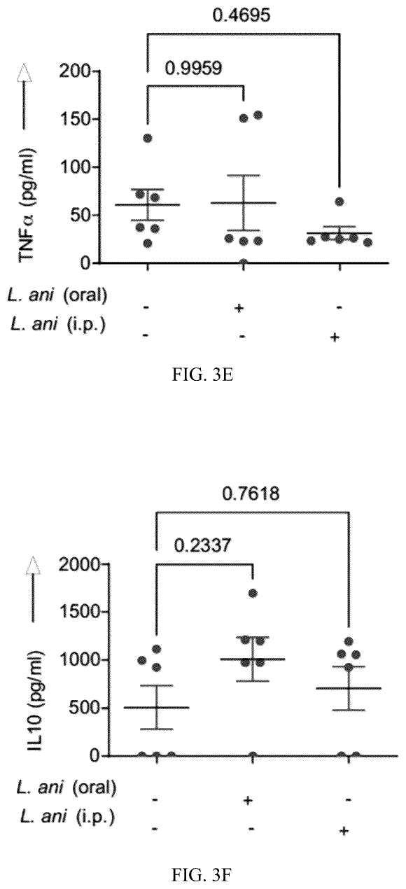

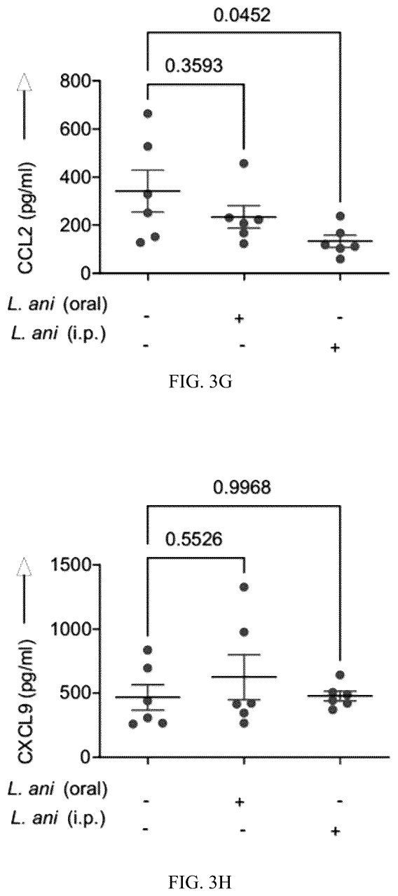

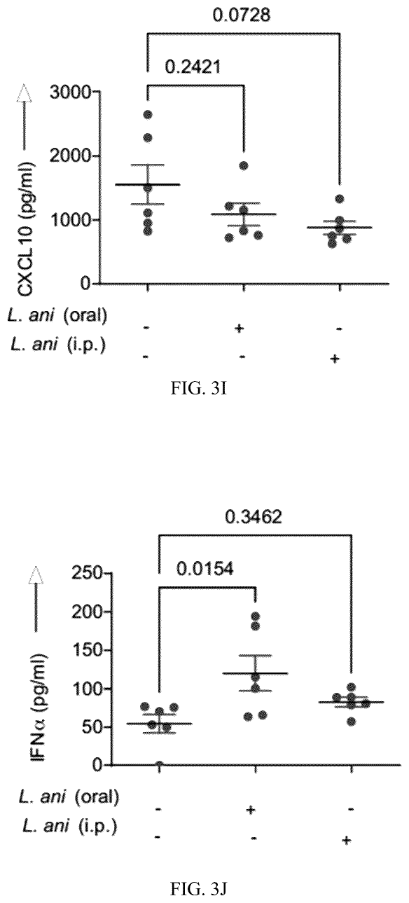

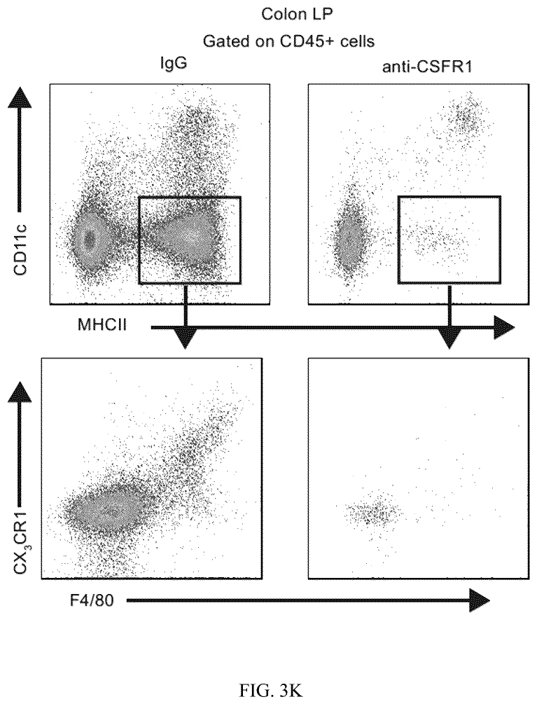

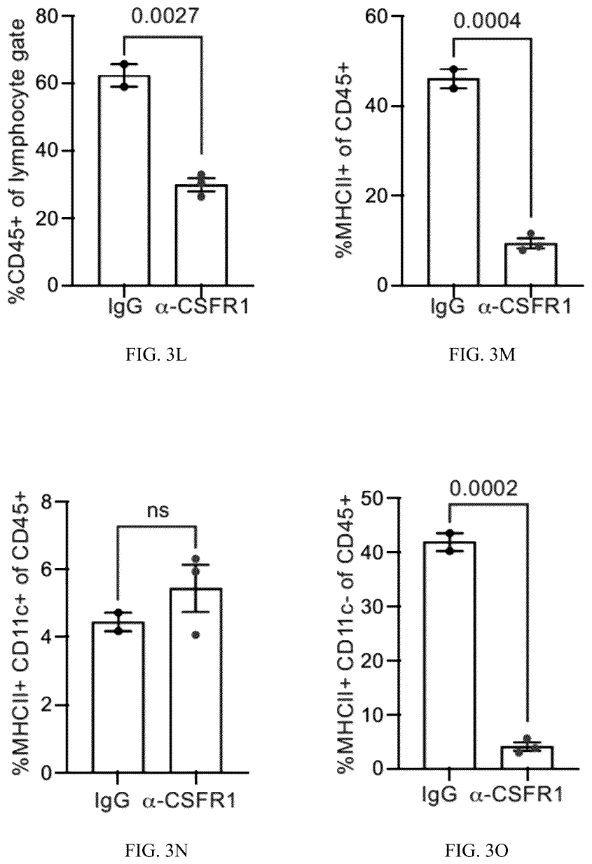

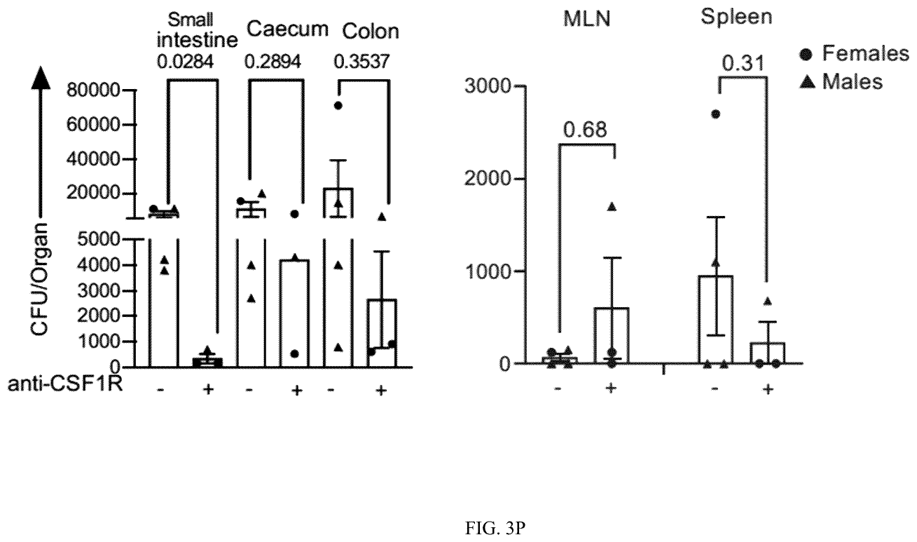

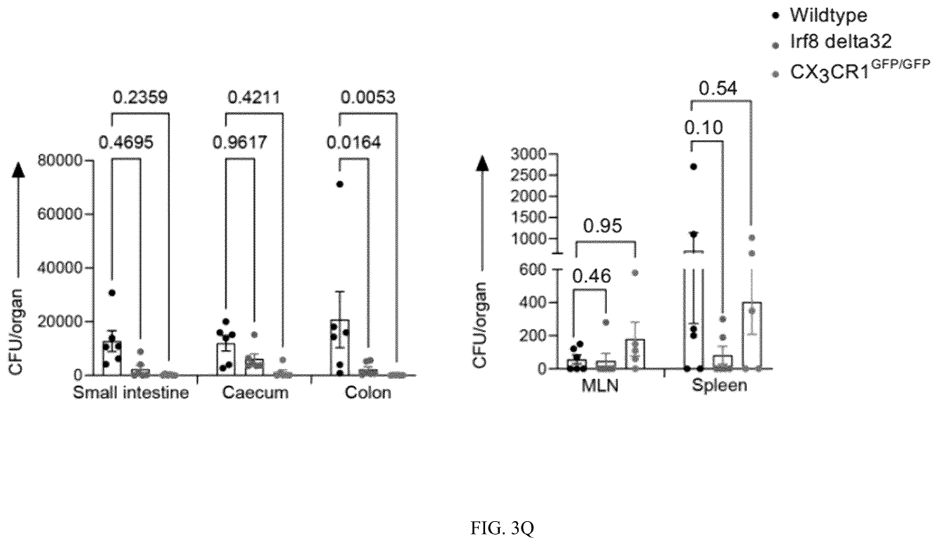

Sphingosine-1-phosphate receptor (S1PR) is a cell surface receptor required for antigen presenting cells (APCs) acquiring luminal antigens from the majority of colonic GAPs to traffic to the draining lymph nodes (Matloubian et al., Nature, 2004, 427, 355-360). Some gut bacteria can survive within lamina propria dendritic cells (Fung et al., Immunity, 2016, 44, 634-646 and Udayan et al., Gut microbes, 2022, 14, 2007743), and S1PR is required for trafficking of some pathogenic bacteria to lymph nodes (St John et al., Immunity, 2014, 41, 440-450). Therefore, further experiments evaluated if S1PR expressing cells facilitated the dissemination of L. animalis^{WU} to extraintestinal tissues in early life by treating preweaning mice with the S1PR modulator FTY720, which inhibits leukocyte trafficking (FIG. 1I). FTY720 treatment significantly reduced S1PR⁺ cells in spleen but not in the colon lamina propria, consistent with S1PR modulation affecting cell trafficking in preweaning mice (FIG. 2J). FTY720 treatment did not affect L. animalis^{WU} colonization of the gut, but impaired its ability to translocate and disseminate (FIG. 2C), consistent with S1PR expressing cells carrying L. animalis^{WU} to distant sites. Genetic and pharmacologic approaches were employed to identify specific host cell subsets facilitating L. animalis^{WU} translocation. CSFR1 blockade inhibits the development of CSFR1 dependent populations which are largely myeloid derived phagocytes (Louis et al., J Immunol, 2015, 195, 134-144 and Wang et al., Eur J Immunol, 2016, 46, 2454-2466). CSF1R blockade depleted CD45+MHCII+CD11c− populations in the preweaning colon that were largely CX₃CR1−F4/80− or CX₃CR1+F4/80+ but did not affect CD45+MHCII+CD11c+ cells (FIGS. 3K-3O). CSF1R blockade reduced L. animalis^{WU} colonization in the small intestine but did not affect translocation and dissemination (FIG. 3P). It was observed that despite lower colonization of L. animalis^{WU} in the colon, the lack of cDC1s in preweaning mice (Irf8 delta+32 enhancer) (Durai et al., Nat Immunol, 2019, 20, 1161-1173) did not impact dissemination of L. animalis^{WU} (FIG. 3Q). Deletion of CX₃CR1 has been observed to inhibit dissemination of gut bacteria in adult mice (Niess et al., Science, 2005, 307, 254-258 and Knoop et al., Gut, 2016, 65, 1100-U1160), however surprisingly CX₃CR1 deficiency did not affect the translocation of L. animalis^{WU} in preweaning mice, despite lower levels of colonization in the colon (FIG. 3Q). Clodronate encapsulated capsule liposomes (Clodrosome) effectively depletes phagocytic cell populations (Van Rooijen et al., Journal of Immunological Methods, 1994, 174, 83-93 and Kozicky et al., Methods Mol Biol, 2019, 1960, 101-112) and accordingly Clodrosome treatment effectively depleted CD45+ MHCII+ F4/80+ cellular populations in the spleen of preweaning mice (FIGS. 2D-E) and impaired L. animalis_{WU} translocation to spleen with a trend toward impairment in the MLN (FIG. 2F). Further experiments assessed if L. animalis^{WU} could be found inside host cells by treating MLNs and splenic cellular populations from L. animalis^{WU} fed preweaning mice with gentamicin, which does not penetrate eukaryotic cell membranes and thus selectively kills extracellular but not intracellular bacteria. Gentamicin treatment killed cultured L. animalis^{WU} (FIG. 2G), but L. animalis^{WU} was still recoverable from splenic (FIG. 2H) and MLN (FIG. 2I) cellular populations, suggesting that L. animalis^{WU} was able to reside, to some extent, intracellularly. Together these observations suggest that translocation and dissemination of gut-resident commensal bacteria in early life is a controlled process that is in part facilitated by the host.

### Example 3—Early Life L. animalis^{WU} Translocation does not Trigger Inflammation

Translocation of live gut-resident bacteria to systemic tissues is generally perceived as an unwanted event that occurs under pathologic conditions resulting in breach of the gut barrier and is linked with systemic pathologies detrimental to the host (Knoop et al., Gut, 2016, 65, 1100-1109; O'Boyle et al., Gut, 1998, 42, 29-35; Ribet et al., Microbes Infect, 2015, 17, 173-183; and McPherson et al., Trends Immunol, 2021, 42, 137-150). Supporting this concept, previous work reported that pathologic opening of colonic GAPs in adult mice results in translocation and dissemination of live gut commensal bacteria triggering inflammation (Knoop et al., Gut, 2016, 65, 1100-1109). Further, early-life bacterial translocation of gut resident pathogens is linked with late onset neonatal sepsis (Knoop et al., Proc Natl Acad Sci USA, 2020, 117, 7941-7949; Kleist et al., Nutrients, 2020, 12; and Greenfield et al., Immunohorizons, 2021, 5, 512-522). Since L. animalis^{WU} was recoverable from extraintestinal tissues of healthy, conventionally raised, laboratory mice and its translocation occurred via mechanisms in part dependent upon the host, it was speculated that, unlike the so far studied pathological translocations, the translocation of L. animalis^{WU} might not be harmful but rather potentially beneficial to the preweaning mice.

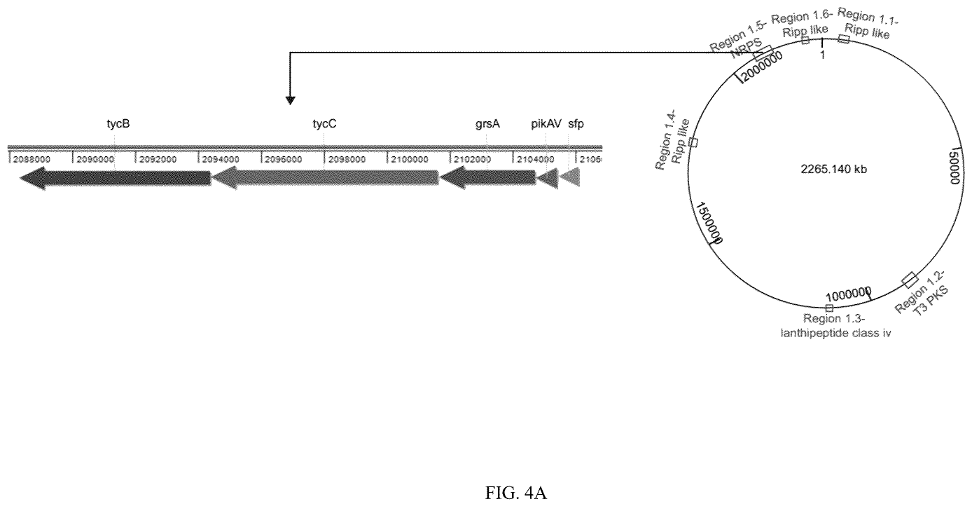

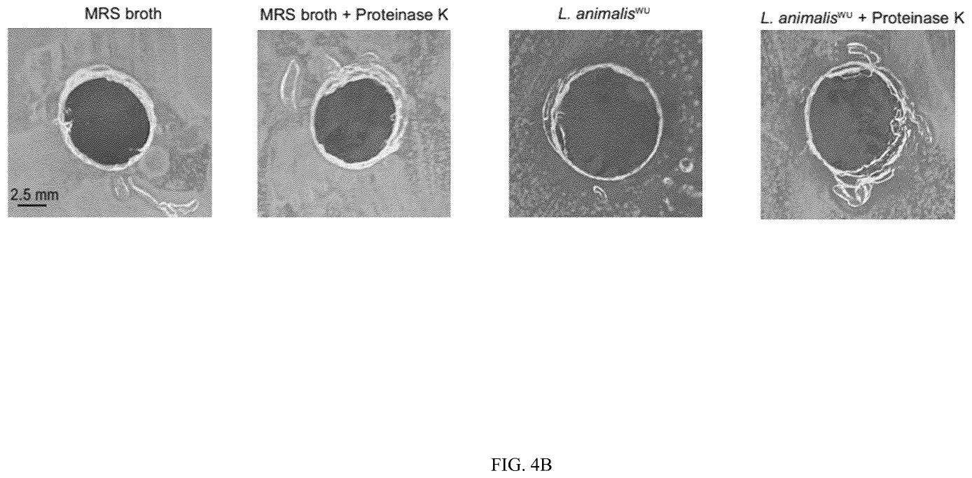

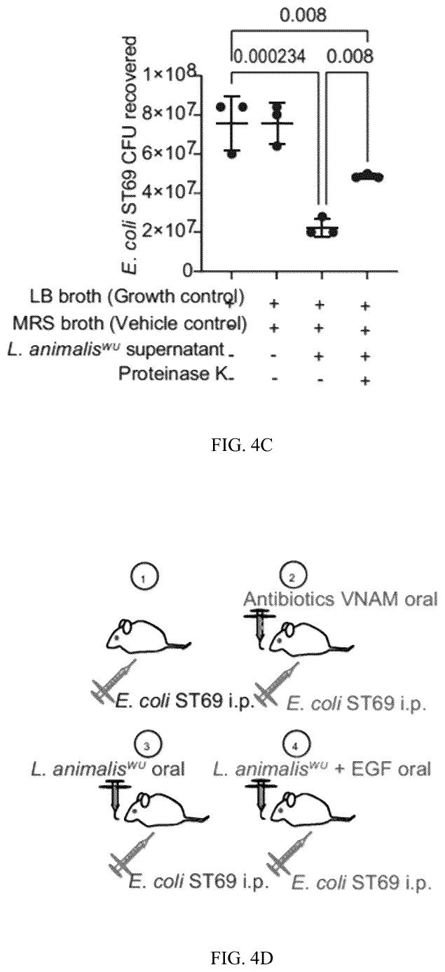

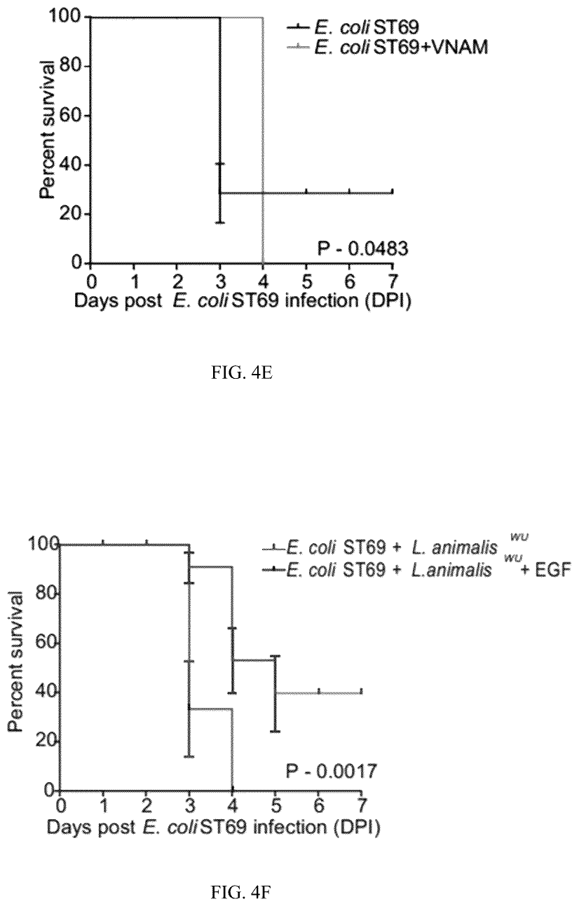

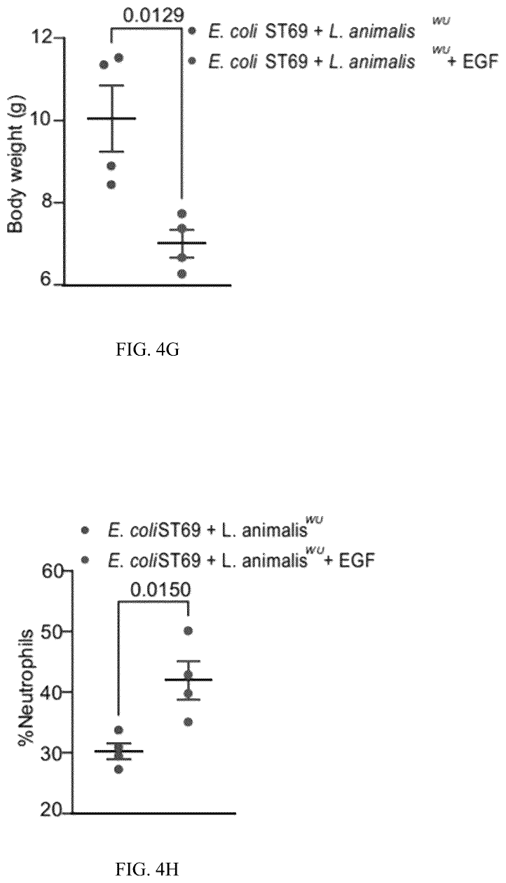

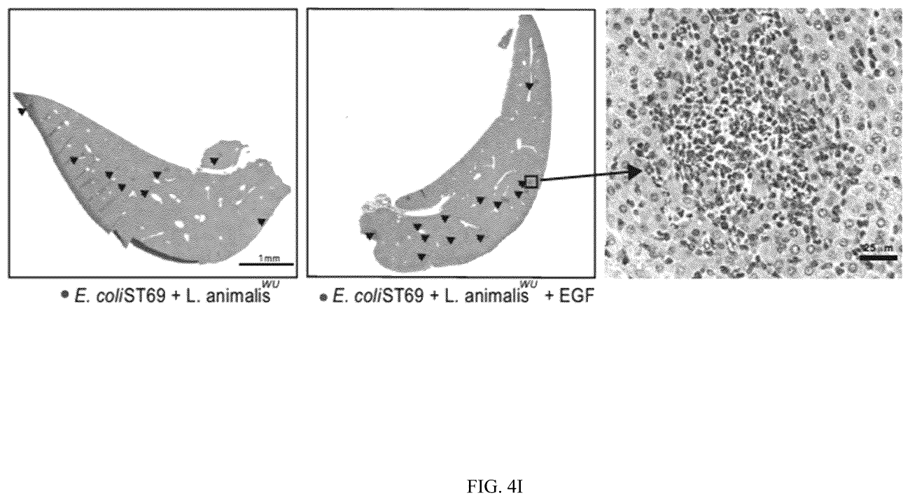

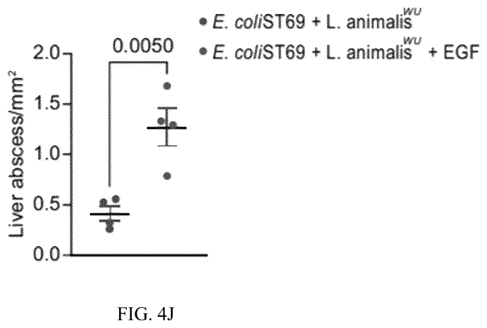

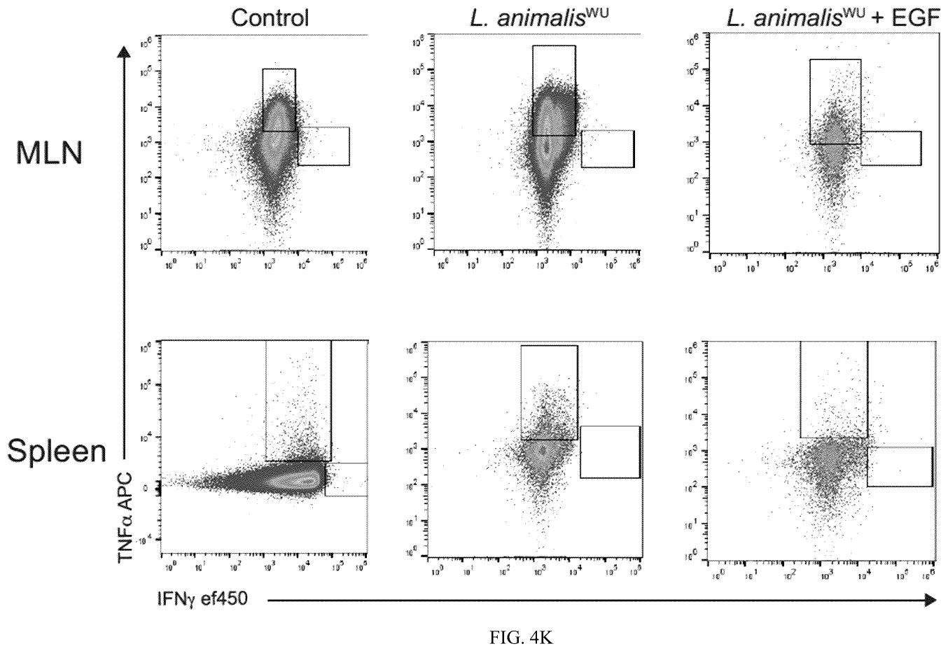

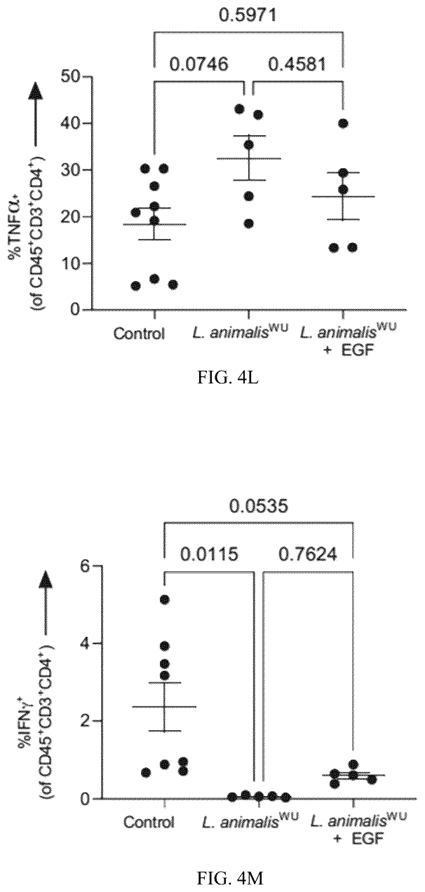

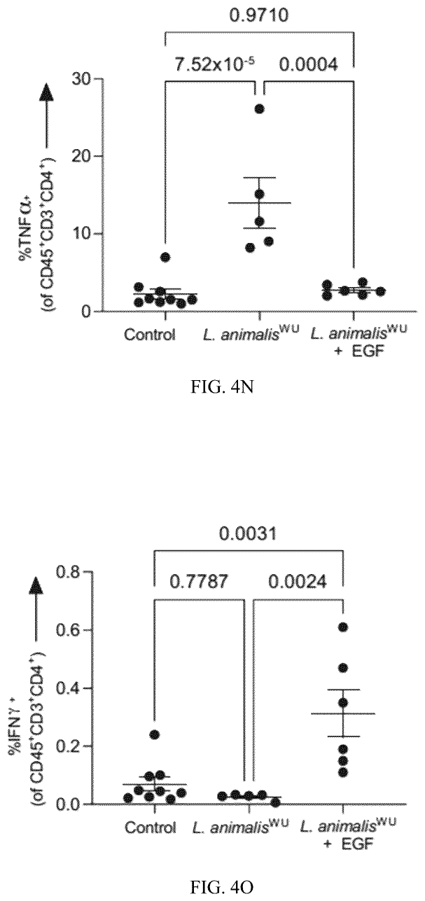

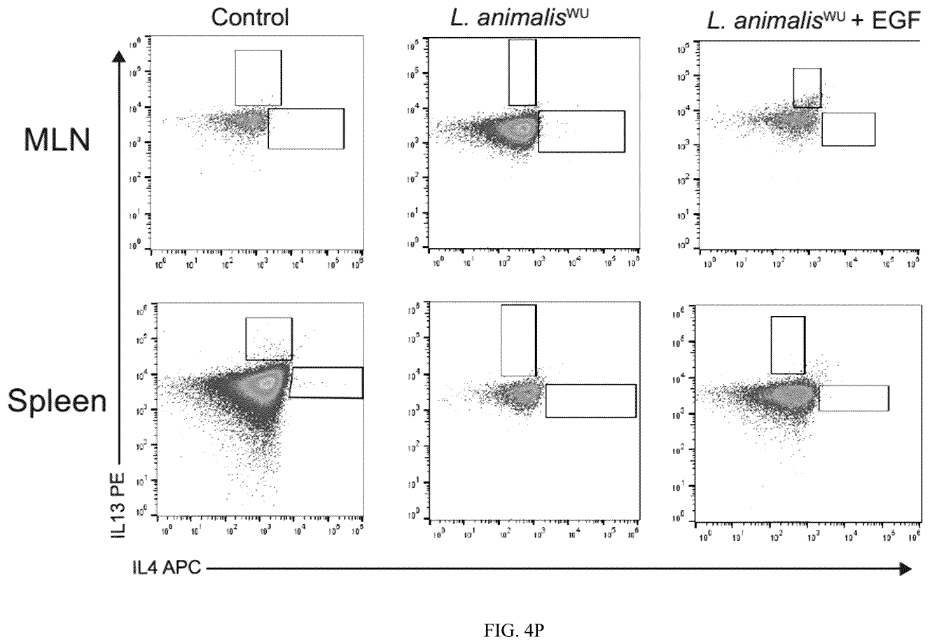

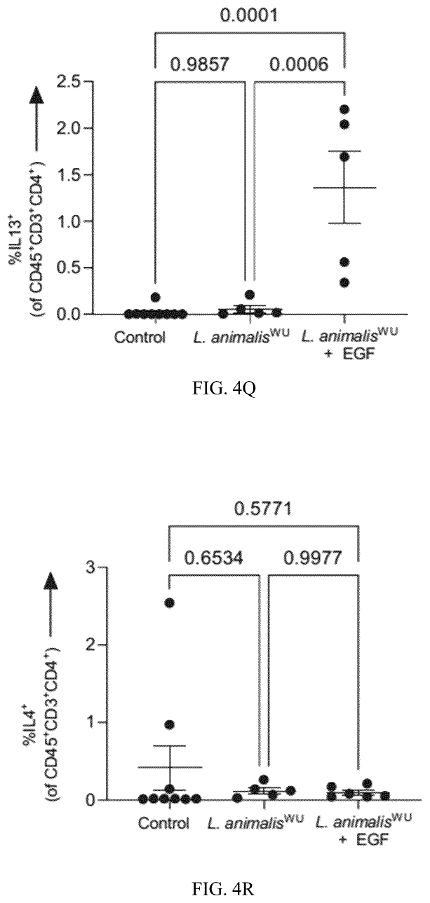

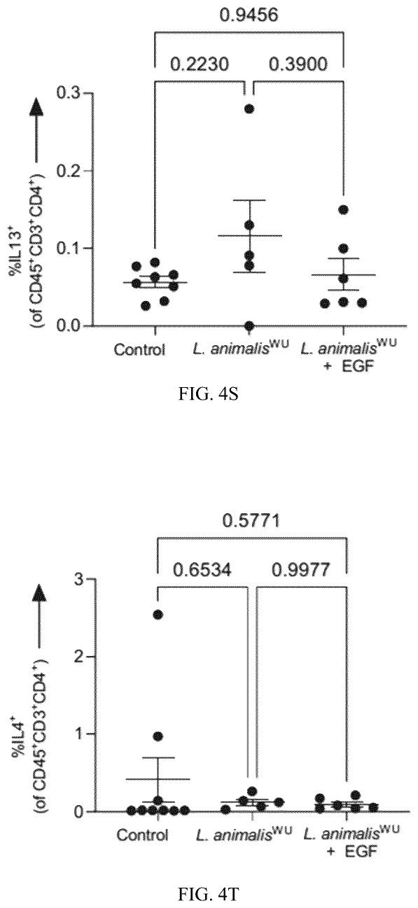

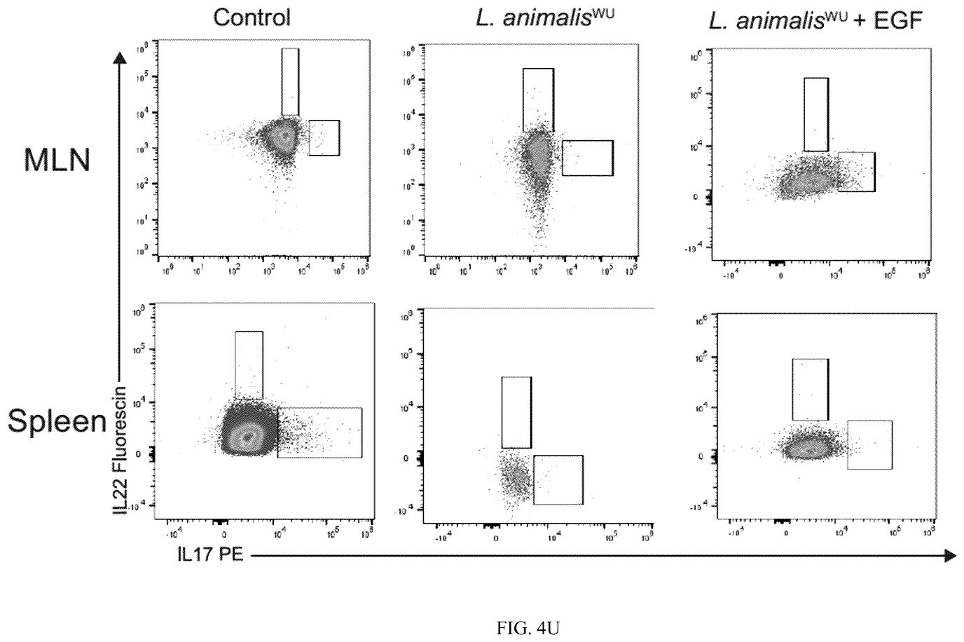

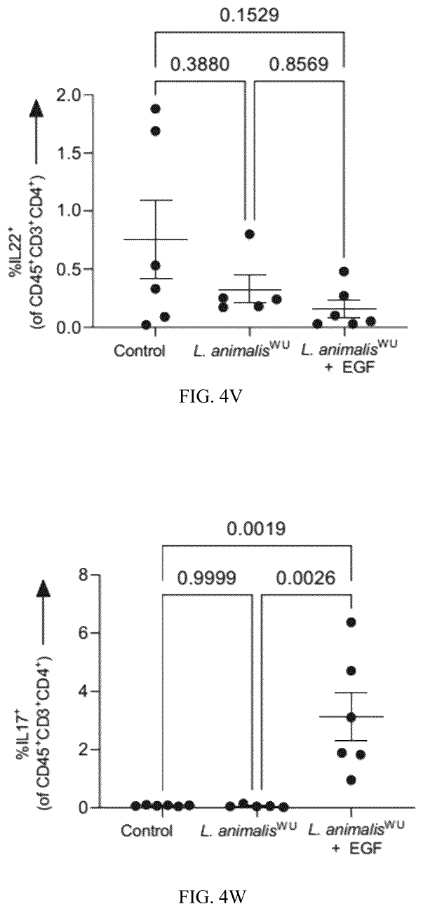

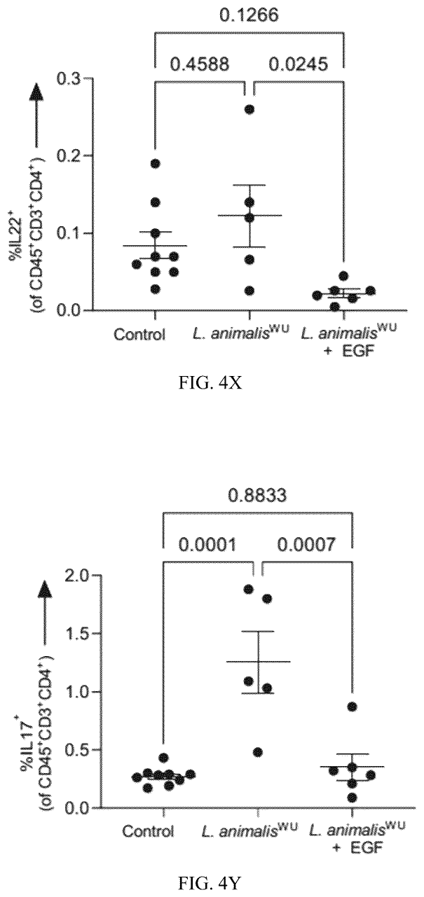

Given that host cells harbor and carry L. animalis^{WU} to distant tissues experiments assessed the effects of translocation on the transcriptome of MLN APCs (CD45⁺CD19⁻B220⁻MHCII⁺) from preweaning mice fed L. animalis^{WU} (confirmed translocation positive) or fed L. animalis^{WU} and EGF (confirmed translocation negative) (FIG. 3A). Surprisingly, despite the presence of live bacteria in the translocation positive group and lack of live bacteria in the translocation negative group, only 69 differentially expressed transcripts were detected (FIG. 3B) which were largely not associated with immune signaling/inflammatory pathways expected in tissues/cells harboring live bacteria. Genes associated with regulation of intracellular signaling cascades (Abl1, Ccdc125, Hmgcr, Nucb2, Pdpk1, Prxl2c, Sos2 and Usp7) were upregulated and genes associated with cell junction organization (Hip1r, Myo1c, Ptpra, Rhob, Smad7, Tjp1 and Zfp703) were downregulated in the translocation positive group (FIG. 3B). Pathway analysis revealed 22 pathways that were enriched (P<0.05), none of which were related to infection or met the criteria of FDR<0.05. Furthermore, blood neutrophils (FIG. 3C), serum cytokines (IL6, TNFα, IL10; FIGS. 3D-F), and chemokines (CCL2, CXCL9 and CXCL10 FIGS. 3G-I) were not different between age-matched control preweaning mice, mice fed L. animalis^{WU} or mice injected with L. animalis^{WU} intraperitoneally (i.p.). IFNα was the only upregulated cytokine seen in mice fed with L. animalis^{WU} orally (FIG. 3J). Further testing assessed if L. animalis^{WU} translocation induced the production of Th1, Th2, or Th17 related cytokines by CD4⁺ T cells in MLNs and spleen (FIGS. 4K-4Y). T cells expressing multiple cytokines related to a T helper subset was not observed. However, significantly increased expression of TNFα and IL17 secretion by splenic CD4⁺ T cells in L. animalis^{WU} colonized mice that was reversed by EGF treatment was observed; this was not seen in MLN CD4+ T cells (FIG. 4N and FIG. 4Y compared with FIG. 4L and FIG. 4W). Interestingly inhibition of translocation by EGF increased production of IL17 by MLN CD4+ T cells (FIG. 4W). This suggests that in early life translocation is detected, or affects, to some extent, the adaptive immune system, however, the responses may vary by organ and not be as robust as expected in adults. The lack of systemic inflammatory response was not universal to all bacteria, as preweaning mice gavaged with E. coli ST69, a pathogen isolated from the blood of a child with late onset sepsis (LOS), displayed neutrophilia (FIG. 5).

### Example 4—Early Life Translocation Protects Against Systemic Infection

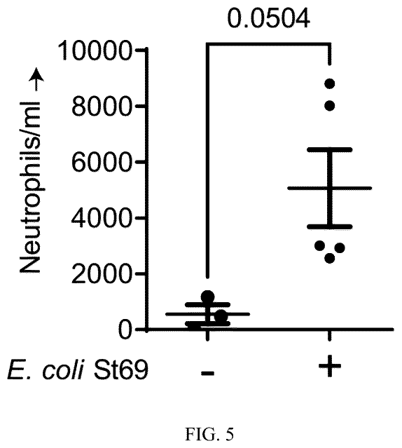

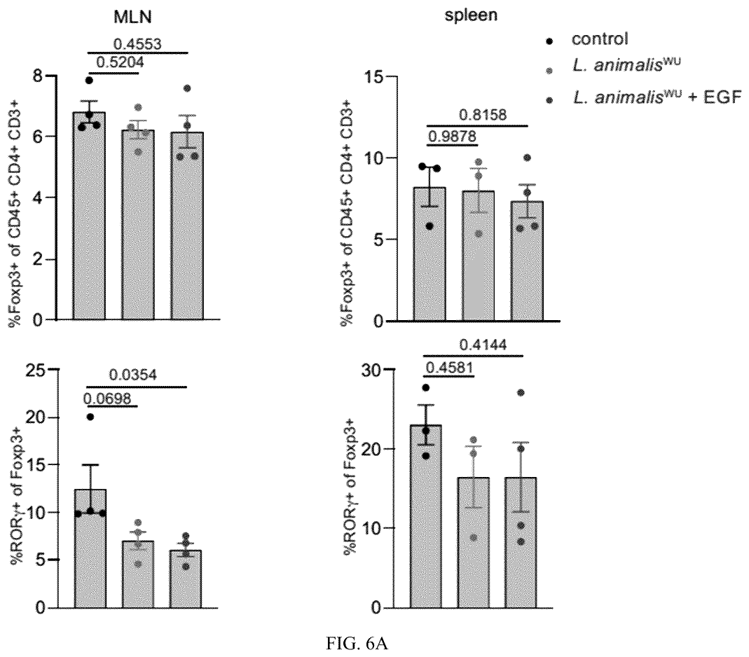

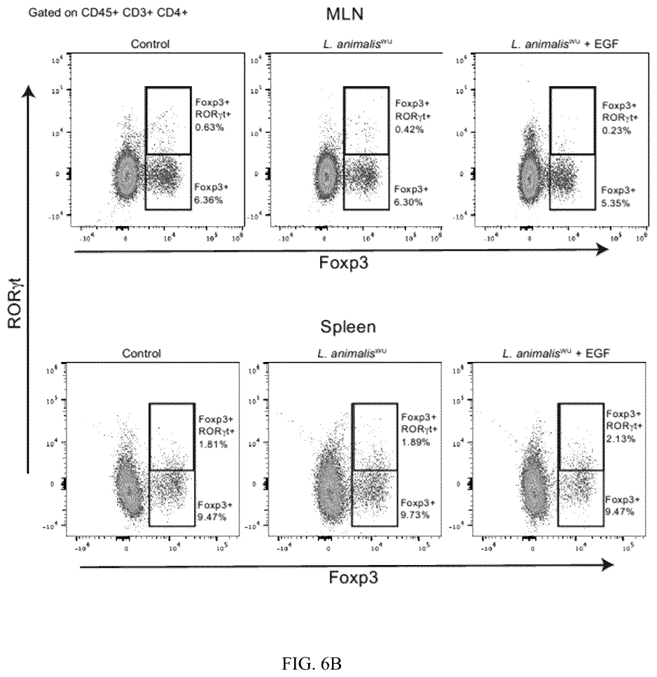

Gut commensal bacteria are associated with immunomodulatory capabilities (Round et al., Nat Rev Immunol, 2009, 9, 313-323), particularly with the induction of RORγt expressing colonic T regulatory cells (Tregs). Induction of colonic RORγt+ Tregs in early life has been attributed to short chain fatty acid (SCFA) producing bacterial taxa (Sefik et al., Science, 2015, 349, 993-997), which largely do not include Lactobacilli. However, L. rhamnosus has been observed to induce colonic RORγt Tregs to some degree (Sefik et al., Science, 2015, 349, 993-997). Consistent with the transcriptomic analysis, gavage of L. animalis^{WU} from DOL10-20 did not increase the frequency of Tregs or RORγt+ Tregs in the spleen or MLN (FIGS. 6A and 6B), however inhibition of GAPs and translocation with EGF reduced the population of RORγt+ Tregs in the MLN, consistent with prior observations of colonic GAP manipulation on colonic RORγt+ Treg populations in preweaning mice (Knoop et al., JCI Insight, 2020, 5). This suggests that Treg induction is not the major beneficial effect L. animalis^{WU} translocation is providing to the host at this time in life.

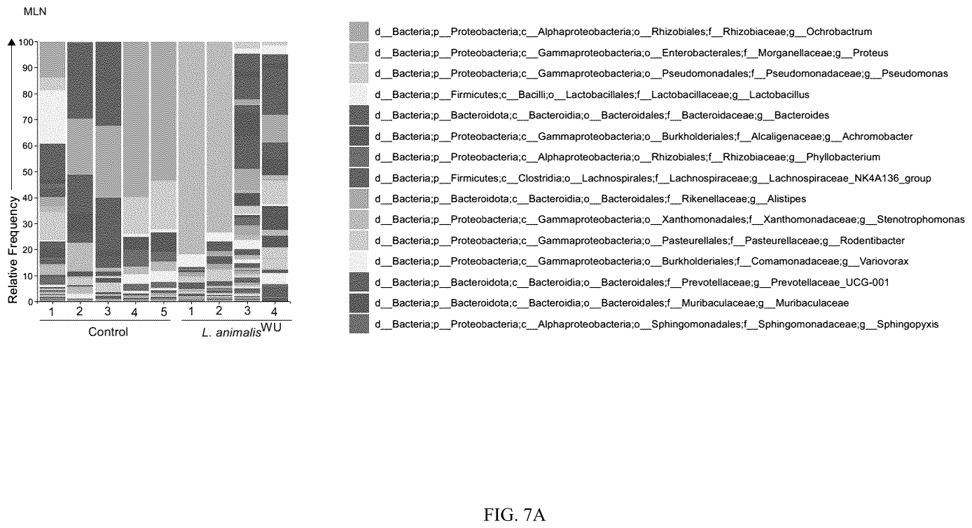

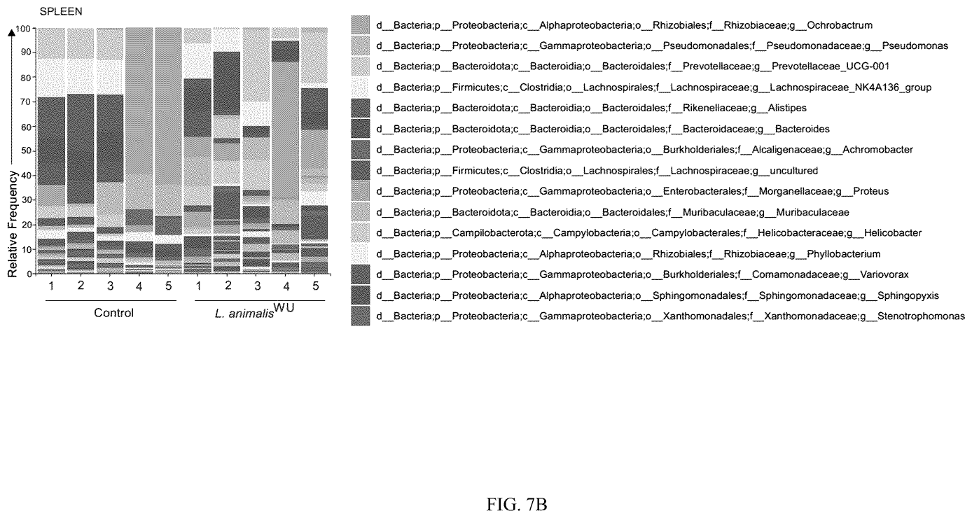

Since these observations suggested that L. animalis^{WU} translocation was physiologic, yet not associated with overt immunological effects on the host, further investigations sought to assess bacteria intrinsic properties that could be beneficial to the host. Sequencing and alignment of the L. animalis^{WU} genome using Prokka identified coding sequences (CDS) for a putative gramicidine-tyrocidine antibiotic synthesizing gene cluster (tycB, tycC, grsA, pikAV and sfp), resembling the organization of an operon (FIG. 4A). Further analysis with antiSMASH (Blin et al., Nucleic Acids Research, 2023, 51, W46-W50) revealed five additional regions putatively coding for genes synthesizing antimicrobial products. These gene clusters were not observed in publicly available databases for Lactobacilli, including L. animalis, and therefore appeared to be a unique feature of this strain. It was then questioned if L. animalis^{WU} translocation might confer antimicrobial, or other potential benefits, in extraintestinal tissues which could limit systemic bacterial infections and/or septic responses in early life when the immune system is not fully developed. To test this concept, an E. coli ST69 was utilized (Carl et al., Clin Infect Dis, 2014, 58, 1211-1218), as previously demonstrated it causes disease in a preweaning mouse model of LOS (Knoop et al., Proc Natl Acad Sci USA, 2020, 117, 7941-7949). L. animalis^{WU} culture supernatants exhibited antibacterial activity against E. coli ST69 in vitro, which was reduced by treatment with proteinase K, suggesting the presence of L. animalis^{WU} protein products conferring antimicrobial activity (FIGS. 4B and 4C). Culture independent approaches that exclude free DNA within the tissue (Nocker et al., Journal of Microbiological Methods, 2006, 67, 310-320 and Rawsthorne et al., Appl Environ Microbiol, 2009, 75, 2936-2939) did not demonstrate quantitative changes in the bacterial taxa in the MLNs of mice receiving augmented L. animalis colonization (FIGS. 7A and 7B), likely reflecting selection/evolution of the early life gut microbial community to co-exist with taxa possessing these properties.

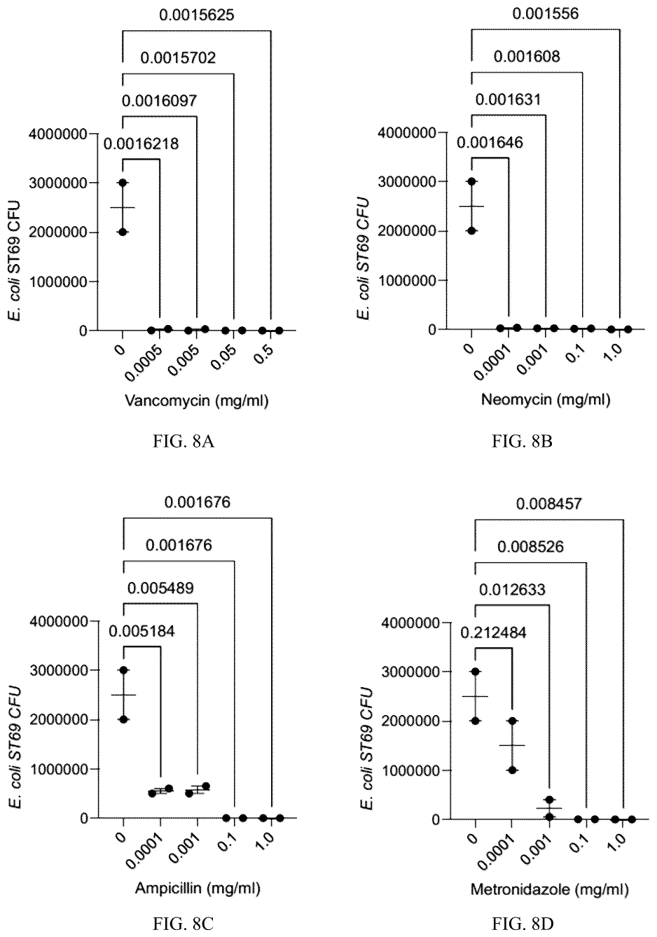

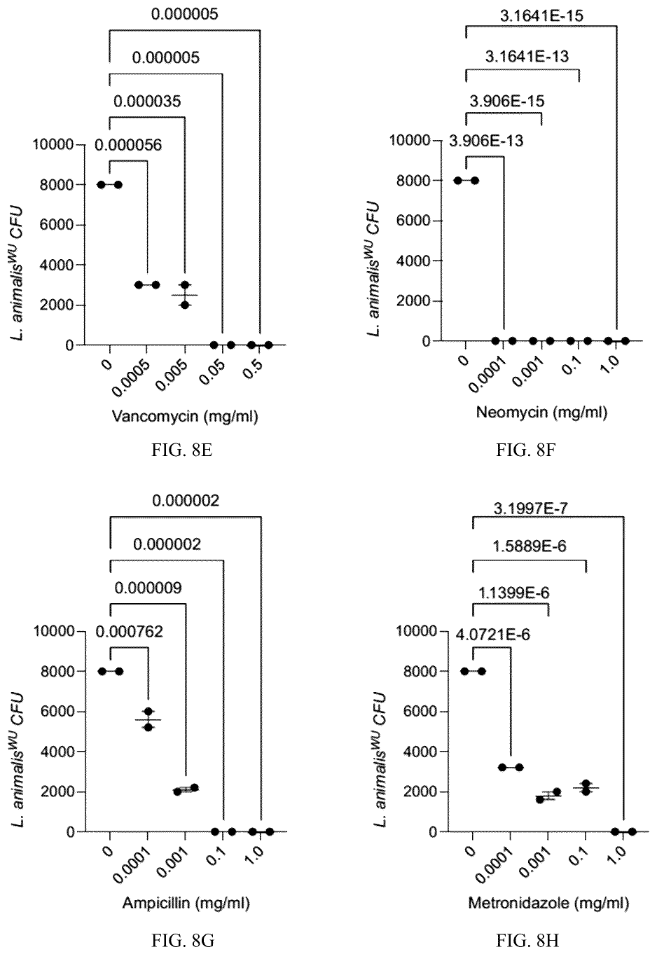

To test if the early-life endogenous gut microbiota, which contains L. animalis^{WU}, is protective against systemic E. coli ST69 infection in vivo, control and oral antibiotic (vancomycin, neomycin, ampicillin, metronidazole) treated preweaning mice were given systemic E. coli ST69 (FIG. 4D). In contrast to the previously described LOS model (Knoop et al., Proc Natl Acad Sci USA, 2020, 117, 7941-7949), E. coli ST69 was given interperitoneally (i.p.) to avoid effects of early life gut microbes on the growth of E. coli ST69 in the gut lumen. Mortality of antibiotic treated mice was delayed by one day, which might reflect presence of residual systemic antibiotics with efficacy to ST69 (FIGS. 4A-4J and FIGS. 8A-8H). However, antibiotic treatment paradoxically made preweaning mice more susceptible, rapidly succumbing to systemic infection despite these antibiotics having activity against ST69 (FIG. 4E). Notably L. animalis^{WU} displayed sensitivity to these antibiotics as well (FIGS. 8A-8H). These observations are consistent with the endogenous gut microbiota, containing L. animalis^{WU}, being protective against systemic E. coli ST69 induced mortality in preweaning mice. To evaluate if the protective effect was further enhanced by supplementing with L. animalis^{WU} and if the protection was reversed by inhibiting translocation, mice were colonized with L. animalis^{WU} with or without EGF treatment to inhibit translocation. Inhibition translocation by EGF treatment increased mortality when compared to mice treated with L. animalis^{WU} alone (FIG. 4F). Mice succumbed to E. coli ST69 with little forewarning, such that attempts to quantitate live pathogen burden in the systemic tissues while the mice were alive were unsuccessful. Therefore, mice were evaluated by necropsy 2.5 days following E. coli ST69 infection, a time in which all mice given L. animalis^{WU} with or without EGF were alive. Mice given E. coli ST69 i.p., L. animalis^{WU}, and EGF had significantly reduced body weight (FIG. 4G). While mice in both groups displayed neutrophilia, mice treated with EGF had a significantly increased percentage of peripheral blood segmented neutrophils consistent with a more severe infection (FIG. 4H). There were no significant gross findings in mice in either group. Histologic examination revealed no significant abnormalities in the brain, heart, large intestine, pancreas, and kidneys, mild enteritis of the small intestine, and mild to moderate extramedullary hematopoiesis in the spleen of mice in both groups. Moderate neutrophilia was seen in the lungs of two mice not receiving EGF. However, mice given E. coli ST69 i.p., L. animalis^{WU} and EGF had significantly increased number of hepatic abscesses (FIGS. 41 and 4J), consistent with worse control of systemic E. coli ST69 infection, or the response to the infection, when translocation was inhibited. In further support of L. animalis^{WU} providing protection, mice receiving augmented L. animalis^{WU} colonization showed enhanced survival during E. coli ST69 infection when compared with unmanipulated mice (compare black line FIG. 4E with blue line FIG. 4F, P=0.0190 (Log-rank Mantel-Cox test)). Therefore, the endogenous preweaning microbiota, and specifically L. animalis^{WU}, enhanced survival to systemic E. coli ST69 infection and this effect was reversed by inhibiting physiologic translocation of resident gut microbes during this preweaning period, however the definitive mechanism(s) behind this benefit (antimicrobial activity vs other effects) remain to be explored.

### Example 5—Colonic Goblet Cell-Associated Antigen Passages Mediate Physiologic and Beneficial Translocation of Live Gut Bacteria in Preweaning Mice

There is a growing appreciation that the gut microbiota is not merely a passive resident but a large contributor to host physiology. Beyond gut centric effects, the gut microbiota has been implicated in disease, and by extension health, in distant organs systems including the endocrine, dermatologic, neurologic, pulmonary, renal, and reproductive systems. Current concepts are that gut residing microbes mediate these extraintestinal events by imprinting local cellular populations which migrate to extraintestinal sites and/or microbial products produced within the gut diffuse to distant sites (Belkaid et al., Immunity, 2017, 46, 562-576). While these concepts are sufficient to explain many, if not most, of the distant effects attributed to the gut microbiota, some observations extraintestinal events mediated by rare and/or unstable gut microbial products (Constantinides et al., Science, 2019, 366) seem at odds with this model.

The instant disclosure identified that select live gut resident bacteria have the capacity to translocate and disseminate in early life. This phenomenon was not dependent upon litter or sex but was dependent upon age. Translocation in early life appeared stochastic with a binary distribution, either occurring with hundreds to thousands of live bacteria translocating or no live bacteria translocating despite equivalent levels of gut colonization by translocating bacteria. While the basis for this pattern of translocation is not completely understood, the impression, based on the timing of colonic GAP formation in early life and observations in mice within or outside of this period of colonic GAP formation, is that translocation occurs during a ~10 day preweaning window and may have missed translocation in some DOL17 mice. Other possibilities such as alterations in the developing microbiota, alterations in maternal or other environmental factors are also potential explanations. However, translocation varied within litters, which have similar exposure to maternal and environmental factors and similar gut microbiotas. Further there were no observed differences in the gut microbiota from mice with and without translocation within the same litter, albeit analysis used 16S rRNA variable region sequencing, which may not have granularity to decipher subtle differences. Consistently it was observed that only a subset of the gut resident bacteria translocate in early life, which was not related to relative abundance with the gut. Lactobacilli were the most common translocating taxa in these studies, yet Lactobacilli made up ~5% of the taxa identified in the preweaning gut microbiota. It is not favored that this is solely a result of failure to culture some taxa, as taxa, which was observed not to translocate, should be culturable by the methods. Based upon observations here and other work it is believed translocation is a property of non-adherent bacteria that can reside in the mucus layer. Other factors that account for the capacity to translocate is unclear but the subject of ongoing work. Likewise, determination of the preferential tissue niche(s) and function(s) of other physiologically translocating species and their effects on the host will require further study.

Translocation and dissemination of gut resident bacteria is almost universally viewed as an unwanted event with detrimental outcomes for the host. Here it was observed that translocation of live gut resident commensal bacteria did not adversely affect the host's health. Moreover, this process was in part host dependent as it required host goblet cells, the formation of GAPs, S1PR function, and phagocytic cells for translocation and dissemination. In contrast to what has been described in adult mice (Niess et al., Science, 2005, 307, 254-258 and Knoop et al., Gut, 2016, 65, 1100-U1160), the current results did not find that dissemination was dependent upon CX₃CR1, nor was it dependent upon cDC1 DCs, or newly developing CSFR1 dependent cells. This likely reflects unique cell populations inhabiting the gut in early life with differing biology from those in adults (Bain et al., Nat Immunol, 2014, 15, 929-937 and Akagbosu et al., Nature, 2022, 610, 752-760). Further systemic dissemination of live translocating bacteria in early life did not elicit a significant inflammatory response, which is in part due to properties of the bacteria and in part due to age specific features of the host. Together these observations suggest this is a physiologic event, and to be evolutionarily conserved, has some benefit to the host at this time in life. Given the role of gut commensal bacteria in shaping the immune system, it was surprising that no role for translocation of L. animalis^{WU} was observed in immune development, although admittedly the investigations were not exhaustive. Whole genome sequencing of L. animalis^{WU} revealed the presence of a clusters of genes putatively producing antimicrobial products, suggesting a bacteria intrinsic purpose for translocation. The regions were not found in publicly available databases of other Lactobacillus species and are therefore inferred to potentially be unique to this strain. L. animalis^{WU} products demonstrated antimicrobial activity in vitro, and in vivo studies demonstrated a benefit of L. animalis^{WU} in a systemic sepsis model, which could be reversed by limiting the translocation of L. animalis^{WU}. Admittedly the studies presented here do not definitively prove this is the mechanism providing protection in this model in vivo. L. animalis^{WU} might have other yet to be discovered properties, potentially including immunomodulation, that benefit the preweaning host during systemic infection.

Lactobacilli spp. have demonstrated antibacterial activity in vitro (Drumond et al., Front Pharmacol, 2023, 14, 1152588) and a Lactobacillus species, L. salivarius UCC118, produces the antibiotic bacteriocin, Abp118, which acts in the gut lumen to protect from infection by the enteric pathogen L. monocytogenes (Corr et al., Proc Natl Acad Sci USA, 2007, 104, 7617-7621). In addition, L. murinus, has also been found to be protective in a model of LOS by acting within the gut lumen to limit pathobiont colonization (Singer et al., Nat Med, 2019, 25, 1772-1782). Thus, although not investigated here, it is likely that L. animalis^{WU} would have role(s) in out competing other microbes and/or conferring antimicrobial activity within the gut lumen which, could likewise confer benefits to the early life host to decrease enteric infections, limit the dissemination of other gut microbes, and facilitate the assemblage of the healthy gut microbial community.

Disclosed herein is a surprising, and by current concepts almost heretical, role for translocation and dissemination of live gut bacteria. Although surprising from today's perspective of live bacterial translocation and septic outcomes, this phenomenon may be less surprising from an evolutionary perspective that suggests endosymbiotic intimate relationships between prokaryotes and their eukaryotic hosts resulted in the development of organelles like mitochondria and chloroplasts (Osteryoung et al., Science, 2003, 302, 1698-1704). Importantly these studies were performed in a limited context regarding host species/strain, housing conditions, and gut microbial community, and thus further investigation will be required to define how far these observations extend in other settings. Logically one speculates if this phenomenon is also true in humans. While a study of random culture of blood and tissue of healthy human infants and monitoring outcomes in the absence of antibiotic therapy, if culture positive, is not possible due to ethical concerns by today's standards, a study six decades ago evaluated random blood cultures from 131 three day old asymptomatic children found ~13% were culture positive without evidence of sepsis or adverse outcomes (Albers et al., The Journal of Pediatrics, 1966, 69, 193-197). This study concluded these positive cultures could result from contamination or transient bacteremia, which were indistinguishable, but might be consistent with physiological translocation in humans in early life.

In summary, the gut microbiota is increasingly appreciated as a major driver of host development and physiology. Current concepts are that benefits of the gut microbes conferred to the host are restricted to microbes residing in the gut lumen and once the gut barrier is breached, microbes have universally pathophysiologic effects on the host. However, the current observations suggest a new level of symbiosis with our gut microbes in early life where translocation and dissemination of live microbes is not an internecine event resulting in mutual destruction, but an unappreciated and intimate level of symbiosis.

### Example 6—Methods

### Mice

Animal procedures and protocols were performed in accordance with ethical and other regulations per the IACUC at Washington University School of Medicine. All mice were on the C57BL/6 background. C57BL/6 mice, S1PR-GFP reporter mice (Jax stock #028623), Math1^{fl/fl} mice (Jax stock #008681), CX₃CR1^{GFP/GFP} mice (Jax stock #005582) were purchased from The Jackson Laboratory (Bar Harbor, ME). Transgenic mice bearing a tamoxifen-dependent Cre recombinase expressed under the control of the villin promoter (vil-Cre-ER^{T2}) mice were a gift from S. Robine (Institut Curie, Paris, France). Irf delta +38 enhancer (cDC1 deficient) mice (Durai et al., Nat Immunol, 2019, 20, 1161-1173) were a gift from Ken Murphy (Washington University in Saint Louis School of Medicine). Generation of Math1^{fl/fl} Vil-^{Cre-ERT2} mice and inducible deletion of GCs by treatment with tamoxifen have been previously described (Knoop et al., Mucosal Immunology, 2015, 8, 198-210). All mice were bred in house for experiments. Mice were housed in a specific-pathogen-free facility and fed routine chow diet. Mice of both sexes were used in this study and where possible littermates were used as controls.

### Bacteria

L. animalis^{WU} were routinely cultured overnight in MRS broth (Thermofisher scientific) and incubated under microaerobic conditions (5% CO₂) at 37° C. E. coli ST69 were routine cultured overnight in LB broth (Fisherscientific) under aerobic conditions (5% CO₂) at 37° C. The bacterial strains were further subcultured as required for experiments.

### Isolation of Mouse Systemic Tissues for the Identification of Gut-Associated Bacteria

Spleens and MLNs were harvested from preweaning mice at day of life (DOL17) and adult mice from the same litter at DOL35. Tissues were homogenized in a Bullet Blender and plated on Luria Broth (LB), Reinforced Clostridium Agar (RCA), Brain Heart Infusion (BHI) or Tryptic Soy Agar (TSA) bacteriological agar plates and incubated under microaerobic conditions (5% CO₂) at 37° C.

Bacterial Identification from Culture Plates by Sanger Sequencing

Colony PCR was carried out on the resulting colonies from plated tissue homogenates, using primers 7F (5′-AGAGTTTGATNNTGGCTCAG-3′; SEQ ID NO: 1) and 1510R (5′-ACGGNTACCTTGTTACGACTT-3′; SEQ ID NO: 2) which target full length of bacterial 16S nRNA gene. PCR products were purified using High Pure PCR product purification kit (Sigma) and identified by full length Sanger-sequencing (Genewiz).

### Deletion of Goblet Cells, Inhibition of GAPs, CSF1-R Blockade, Clodrosome Treatment and S1PR Modulation

For inducible deletion of goblet cells in preweaning mice, Math1f/f and Math1^{f/f}vil^{ERT2-CRE} mice were injected with tamoxifen (30 μg/mice) via intraperitoneal (i.p.) administration route from DOL9 to DOL12. For inhibition of colonic GAPs, preweaning mice were fed orally with EGF (1 μg/mice) from DOL10-DOL16. For inhibition of S1PR+ cell trafficking, mice were administered with FTY720 (Cayman chemical) from DOL12-16 at 5 mg/kg i.p. For depletion of host cells which require CSF1-R receptor signaling for growth, mice were i.p. administered with anti-CSF1-R (clone AFS98; BioXcell) at 37.7 μg/g on DOL10, DOL13 and DOL14. For depletion of phagocytic cells mice were i.p. administered with 50 μl Clodrosome (Encapsula Nanosciences) on DOL13 and DOL14.

L. animalis^{WU} Administration in Mice

For assessing L. animalis^{WU} intestinal colonization and translocation in all mice models used in this study, L. animalis^{WU} transformed with erythromycin resistant plasmid (pLEM415-IdhL-mRFP1, Addgene) were fed orally to preweaning mice at 10⁸ cells of L. animalis^{WU} per mouse per dose, for three consecutive days (DOL13-15 or DOL 31-33) followed by one day of rest before harvesting and plating tissues from mice at DOL17 or DOL35 respectively. Bacterial colonization in intestinal tissues were confirmed by plating tissue homogenates in BHI agar supplemented with enythnomycin (25 μg/ml). In experiments to evaluate the effects of systemic L. animalis^{WU}, L. animalis^{WU} was injected at DOL16 at 10³ cells per mice intraperitoneally (i.p.).

### Barrier Leak Assay

Mice were fed with 10⁸ L. animalis/day (DOL13-15) were fed alone or with 1 ug EGF/day (DOL10-16), and orally administered with 250 ug 4 kD FITC dextran (Chondrex Inc) for 2 hours, serum was collected, and fluorescence measured by spectrophotometry.

E. coli ST69 Infections in Mice

To assess neutrophilia, 10⁸ E. coli ST69 (Carl et al., Clin Infect Dis, 2014, 58, 1211-1218) was fed orally and neutrophils assessed a day later. For assessing protection against systemic infections of E. coli ST69 mice were injected with a single dose of the bacteria via i.p. administration of 10³ cells at DOL16.

### Antibiotic Treatment

Mice were given Vancomycin (250 mg), Neomycin (500 mg), Ampicilin (500 mg), and Metronidazole (500 mg) in 250 ml of drinking water from DOL6 to DOL14.

### In Vitro Antibacterial Activity Assays

For antibacterial agar well diffusion assays overnight culture supernatants from L. animalis^{WU} were untreated or treated with proteinase K at 56° C. for 30 minutes, and incubated on LB agar with E. coli ST69, with supernatants added in a well created by removing agar at the center of the plate. For time-kill assay, overnight culture of E. coli ST69 were subcultured in 5 ml LB broth supplemented with supernatants from L. animalis^{WU} that were untreated or treated with proteinase K for 6 hours, and, then plated to enumerate colonies on the culture plate 24 hours later.

### Neutrophil and T Cell Assessment by Flow Cytometry

For neutrophil assessment, two drops (10 μL) of blood were collected via cheek bleed, directly into a microcentrifuge tube containing 150 μL of 50 mM ethylenediaminetetraacetic acid (EDTA). Neutrophils in blood were identified as CD45⁺ PE-Cyanine7 (monoclonal antibody 30-F11, eBioscience, ThermoFisher Scientific) and Ly6G/Ly6C⁺ APC (monoclonal antibody RB6-8C5, eBioscience). For assessing cytokine induction by CD4+ T cells, MLNs and spleens were stimulated with cell stimulation cocktail (CYTEK) for 2 hours followed by fixation with Intracellular Fixation & Permeabilization Buffer (eBioscience) and staining. For CD4⁺ FOXP3⁺ and CD4⁺ FOXP3⁺RorγT⁺ regulatory T cell subset analysis, single cell suspension of MLN and spleen were immunostained with CD45 PE-Cyanine7 (monoclonal antibody 30-F11, eBioscience, ThermoFisher Scientific), CD4 APC (monoclonal antibody (GK1.5), eBioscience, ThermoFisher Scientific), FOXP3 FITC (monoclonal antibody (FJK-16s), eBioscience, ThermoFisher Scientific) and BV421 anti-mouse Rorγt (BD Horizon™). For Th1, Th2 and Th17 cytokine analysis spleens and MLNs were stained with anti-TNFα APC (monoclonal antibody (MP6-XT22), eBioscience, ThermoFisher Scientific), anti-IFNγ ef450 (monoclonal antibody (XMG1.2), eBioscience, ThermoFisher Scientific), anti-IL13 PE (monoclonal antibody (eBio13A), eBioscience, ThermoFisher Scientific), anti-IL4 APC (monoclonal antibody (11B11), eBioscience, ThermoFisher Scientific), anti-IL22 Fluorescin (monoclonal antibody (140301), Biotechne) and anti-IL17 PE (monoclonal antibody (eBio17B7), eBioscience, ThermoFisher Scientific). Single cell suspensions of all tissues were stained in buffer containing affinity purified human IgG (Innovative Research, Fisher Scientific) to reduce non-specific staining by the target antibodies. Immunostained samples were analyzed on an Attune NXT flow cytometer (Invitrogen). Flow cytometry data was analyzed using FlowJo v10.7.0.

### Bulk RNA Sequencing and Analysis

MLNs were pooled from multiple pups from DOL17 mice litters, CD45+ MHCII+ CD11c+ cellular populations were isolated by flow cytometric sorting and RNA was extracted using RNeasy Plus Mini Kit (Qiagen, Germantown, MD, USA). RNA was quantified using Qubit Fluorometer (Life Technologies, Carlsbad, CA, USA) and RNA integrity was checked with TapeStation (Agilent Technologies, Palo Alto, CA, USA). RNA library preparations and sequencing reactions were conducted at Azenta US, Inc (South Plainfield, NJ, USA). SMART-Seq v4 Ultra Low Input Kit for Sequencing was used for full-length cDNA synthesis and amplification (Clontech, Mountain View, CA), and Illumina Nextera XT library was used for sequencing library preparation. Briefly, cDNA was fragmented, and adaptor was added using Transposase, followed by limited-cycle PCR to enrich and add index to the cDNA fragments. The final library was assessed with Agilent TapeStation. The sequencing libraries were multiplexed and clustered on a flowcell. After clustering, the flowcell was loaded on the Illumina instrument according to manufacturer's instructions. The samples were sequenced using a 2×150 Paired End (PE) configuration. Image analysis and base calling were conducted by the Illumina software. Raw RNA sequence fastq files were quality filtered, adaptors trimmed, aligned via STAR, annotated, normalized and differentially expressed genes identified using DESeq2 with cutoffs of FDR<0.05, P<0.05, and 2 fold change. Analysis and KEGG pathway enrichment was generated in Partek® Flow®.

### Serum Cytokine Assessment by Flow Cytometry

Two drops (10 μL) of blood were collected via cheek bleed directly into a microcentrifuge tube, stored at room temperature for 20 minutes and serum was isolated by centrifugation at full speed for 8 minutes in a microcentrifuge. Cytokines and chemokines were assessed for in the isolated serum samples using Legendplex (Mouse Cytokine Syndrome Panel, 13 plex, Biologend) on an Attune NxT flow cytometer (Invitrogen) and data were analyzed by following manufacturer's instructions.

### Bacterial 16s Sequencing and Analysis

DNA was isolated from colonic contents with the QIAamp Fast DNA Stool Mini kit from stool of DOL 17 or DOL 35 mice. DNA was isolated from MLNs following treatment with 50 μM propidium monoazide (Biotium) and photo-activated for 15 minutes per the manufacturer's recommendations to cross link free DNA (Nocker et al., Journal of Microbiological Methods, 2006, 67, 310-320 and Rawsthorne et al., Appl Environ Microbiol, 2009, 75, 2936-2939) prior to processing. PCR products were gel purified and submitted for the sequencing of the V4 regions of 16s ribosomal RNA. Briefly, quality and concentration of the samples were assessed on an Agilent BioAnalyzer High Sensitivity DNA Chip. Samples were denatured and libraries were sequenced using the MiSeq 500v2 Ilumina kit and 2×250 sequencing depth was achieved on an Illumina MiSeq. QIIME2 version 2023.5 was used to perform quality control and analysis of the 16s V4 sequence reads. Raw fastq files were demultiplexed and denoised with dada2. Taxonomy was assigned to the amplicon sequence variants (ASVs) using the feature classifier classify-consensus-blast against pre-formatted SILVA reference and taxonomy files.

Whole Genome Sequencing of L. animalis^{WU}

The L. animalis^{WU} genomic DNA was extracted using DNeasy® Blood & Tissue kit (Qiagen). Whole genome sequencing was performed using Illumina and ONT at Microbial Genome Sequencing Center (Pittsburgh, PA). Quality control and adapter trimming of the raw sequence reads was performed with bcl2fastq and porechop for Illumina and ONT sequencing respectively. Hybrid assembly with Illumina and ONT reads was performed with Unicycler. Assembly statistics were recorded with QUAST. Assembly annotation was performed with Prokka and antiSMASH.

Infection of Preweaning Mice with E. coli ST69

Control preweaning mice or preweaning mice treated with L. animalis^{WU} (DOL13-15) with/without co-treatment with EGF (DOL10-16) or treated with VNAM antibiotic cocktail as noted above from (DOL6-14), were infected with 10³ cells of E. coli ST69 via i.p. at DOL16 and assessed for survival until weaning.

### Microscopy

For visualization of gut-resident bacteria in colonic goblet cells, proximal colons were isolated from DOL17 mice, fixed in carnoy buffer and embedded in optimal cutting temperature medium (Fisher Scientific). Tissues were sectioned and stained with eubacterial FISH probe (Integrated DNA Technologies) overnight in hybridization buffer (0.9M sodium chloride, 10% SDS, 40% formamide) followed by further staining with DAPI (Invitrogen) and UEA1 (Vector laboratories). For visualization of abscesses in liver from mice infected with E. coli ST69, liver tissues were isolated from euthanized mice, fixed in formalin buffer and paraffin embedded. Tissues were sectioned and stained with hematoxylin and eosin. Images were obtained using a Keyence BZ-X800 microscope.

### Complete Blood Count Analysis

Automated complete blood count analysis and differential counts were obtained by analyzing the blood samples in Hemavet 1700 Multispecies Hematology Analyzer (Drew Scientific, Oxford, CT).

### Statistical Analysis

Statistical analysis for normally distributed data was calculated using a Student's t-test or one way ANOVA in graph pad prism. Statistical analysis for binomially distributed data was calculated using a one sided cumulative binomial distribution probability using the more frequent occurring group as the comparator in Microsoft Excel. P<0.05 was considered significant.

### Data Availability

The stool sequencing data and L. animalis^{WU} whole genome sequencing data are archived on the Sequence Read Archive (BioProject: PRJNA1067122 and PRJNA1066880). The RNA-seq data is archived on Gene Expression Omnibus (GSE278303).

What is claimed is:

1. A method of protecting a baby from sepsis, comprising administering to the gut of the baby a strain of bacteria that translocates from the gut and disseminates to extraintestinal tissue, wherein the bacteria does not induce a significant inflammatory response or a systemic inflammatory response.

2. A method of preventing sepsis in a baby, comprising administering to the gut of the baby a strain of bacteria that translocates from the gut and disseminates to extraintestinal tissue, wherein the bacteria does not induce a significant inflammatory response or a systemic inflammatory response.

3. A method of treating sepsis in a baby, comprising administering to the gut of the baby a strain of bacteria that translocates from the gut and disseminates to extraintestinal tissue, wherein the bacteria does not induce a significant inflammatory response or a systemic inflammatory response.

4. A method of improving the microbiota of a baby, comprising administering to the gut of the baby a strain of bacteria that translocates from the gut and disseminates to extraintestinal tissue, wherein the bacteria does not induce a significant inflammatory response or a systemic inflammatory response.

5. A method of improving the health of a baby, comprising administering to the gut of the baby a strain of bacteria that translocates from the gut and disseminates to extraintestinal tissue, wherein the bacteria does not induce a significant inflammatory response or a systemic inflammatory response.

6. The method of claim 1, wherein the baby is a newborn.

7. The method of claim 1, wherein the baby is up to 2 or 3 months old.

8. The method of claim 1, wherein the baby is up to 6 months old.

9. The method of claim 1, wherein administration of the bacteria to the gut comprises oral administration.

10. The method of claim 1, wherein translocation from the gut comprises Goblet cell-mediated translocation.

11. The method of claim 1, wherein the bacteria comprises a strain of Lactobacillus.

12. The method of claim 11, wherein the bacteria comprises Lactobacillus animalis.

13. The method of claim 12, wherein the strain of bacteria comprises Lactobacillus animalis^{WU}.

14. A composition comprising a strain of bacteria that translocates from the gut and disseminates to extraintestinal tissue, wherein the bacteria does not induce a significant inflammatory response or a systemic inflammatory response, and wherein the composition is formulated for administration to the gut of a baby.

15. The formulation of claim 14, wherein administration is oral administration.

16. The formulation of claim 14, wherein the formulation comprises a foodstuff.

17. The formulation of claim 14, wherein the formulation comprises a buffer, a salt, or a sugar.

18. The formulation of claim 14, wherein the bacteria is lyophilized (freeze-dried).

19. The formulation of claim 14, wherein translocation of the bacteria comprises Goblet cell-mediate translocation.

20. The method of claim 14, wherein the bacteria comprises a strain of Lactobacillus.

21. The method of claim 20, wherein the bacteria comprises Lactobacillus animalis.

22. The method of claim 21, wherein the strain of bacteria comprises Lactobacillus animalis^{WU}.
